(19) United States
(12) Patent Application Publication
(10) Pub. No.: US 2010/0179122 A1
(43) Pub. Date: Jul. 15, 2010

(54) Non-Nucleoside Reverse Transcriptase Inhibitors

(76) Inventors: Craig W. Lindsley, Schwenksville, PA (US); William H. Leister, Quakertown, PA (US); Scott E. Wolkenberg, Jenkintown, PA (US)

Correspondence Address: MERCK, P O BOX 2000, RAHWAY, NJ, 07065-0907, US

(21) Appl. No.: 11/922,681

(22) PCT Filed: Jun. 23, 2006
(86) PCT No.: PCT/US2006/024611

§ 371 Date: Dec. 20, 2007

Related U.S. Application Data
(60) Provisional application No. 60/694,744, filed on Jun. 28, 2005.
(60) Provisional application No. 60/707,365, filed on Aug. 11, 2005.

(51) Int. Cl. A61K 31/404; C07D 413/12; A61K 31/5377; C07D 209/40; C07D 405/12; C07D 401/12; A61K 31/4439; A61K 31/454; A61K 31/427; C07D 417/12; C07D 401/14; A61K 31/496; A61P 31/18

(52) U.S. Cl. 514/210.21; 544/144; 514/235.2; 514/418; 548/484; 548/454; 514/414; 546/277.7; 514/339; 546/201; 514/323; 514/365; 548/181; 544/364; 514/253.09

(57) ABSTRACT

Compounds of Formula (I): are HIV reverse transcriptase inhibitors, wherein R¹, R², R³, R⁴ and R⁵ are defined herein. The compounds of Formula (I) and their pharmaceutically acceptable salts are useful in the inhibition of HIV reverse transcriptase, the prophylaxis and treatment of infection by HIV and in the prophylaxis, delay in the onset, and treatment of AIDS. The compounds and their salts can be employed as ingredients in pharmaceutical compositions, optionally in combination with other antivirals, immunomodulators, antibiotics or vaccines.

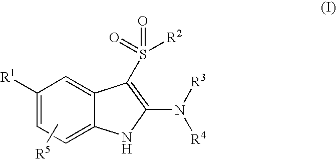

### FIELD OF THE INVENTION

The present invention is directed to certain indoles and their pharmaceutically acceptable salts and their use for the inhibition of HIV reverse transcriptase, the prophylaxis and treatment of HIV infection and HIV replication, and the prophylaxis, delay in the onset of and treatment of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme know as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are the RT inhibitors 3′-azido-3′-deoxythymidine (AZT), 2′,3′-dideoxyinosine (MI), 2′,3′-dideoxycytidine (ddC), d4T, 3TC, nevirapine, delavirdine, efavirenz and abacavir.

While each of the foregoing drugs is effective in treating HIV infection and AIDS, there remains a need to develop additional HIV antiviral drugs including additional RT inhibitors. A particular problem is the development of mutant HIV strains that are resistant to the known inhibitors. The use of RT inhibitors to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a particular need for new RT inhibitors that are effective against mutant HIV strains.

The following references are of interest as background:

U.S. Pat. No. 4,654,360 discloses certain 3-phenylsulfinylindoles and 3-phenylsulfonylindoles to be lipoxygenase inhibitors suitable for the treatment of inflammation.

Williams et al., J. Med. Chem. 1993, vol. 36, pp. 1291-1294 discloses 5-chloro-3-(phenylsulfonyl)indole-2-carboxamide as a non-nucleoside inhibitor of HIV-1 reverse transcriptase.

Young et al., Bioorg. & Med. Chem. Letters 1995, vol. 5, pp. 491-496 discloses certain 2-heterocyclic indole-3-sulfones as inhibitors of HIV-1 reverse transcriptase.

GB 2,282,808 discloses certain 3-substituted heterocyclic indoles as inhibitors of HIV reverse transcriptase and its resistant varieties.

Takahashi et al., Synthesis 1998, no. 7, pp. 986-990 discloses 2-anilino-5-chloro-3-phenylsulfonyl-1H-indole and 2-anilino-5-chloro-3-(4-methylphenyl)sulfonyl-1H-indole.

WO 02/083216 A1 and WO 2004/014364 A1 each disclose certain substituted phenylindoles for the treatment of HIV.

U.S. Pat. No. 5,190,968; U.S. Pat. No. 5,204,344; U.S. Pat. No. 5,252,585; U.S. Pat. No. 5,272,145; U.S. Pat. No. 5,273,980; U.S. Pat. No. 5,290,798; U.S. Pat. No. 5,380,850; and U.S. Pat. No. 5,389,650 disclose certain indoles as inhibitors of leukotriene biosynthesis.

WO03/099206 A2 discloses certain 2-substituted 5-oxazolyl indole compounds useful as inhibitors of IMPDH enzyme.

US 2003/0078288 A1 discloses certain indole derivatives having certain substituted phenyl groups attached to the 5-position of the indole ring via O, S, S(O), S(O)₂, CH₂, CHF, CF₂, NH, or N(C₁₋₄ alkyl). The derivatives are said to be useful for treating all indications which can be treated with natural thyroid hormones.

US 2003/0195244 A1 discloses certain indole compounds having anti-cancer activities, including certain compounds having (3,4,5-trimethoxyphenyl)sulfonyl or (3,4,5-trimethoxyphenyl)carbonyl substituted at the 3-position of the indole ring.

### SUMMARY OF THE INVENTION

The present invention is directed to certain indole compounds and their use in the inhibition of HIV reverse transcriptase, the prophylaxis of infection by HIV, the treatment of infection by HIV, and the prophylaxis, treatment, and delay in the onset of AIDS and/or ARC. More particularly, the present invention includes compounds of Formula I and pharmaceutically acceptable salts thereof:

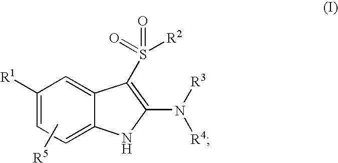

wherein:

### R¹ is:

(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)N(R^{A})R^{B},
(17) N(R^{A})C(O)N(R^{A})R^{B},
(18) C₁₋₆ alkyl,
(19) C₁₋₆ haloalkyl,
(20) C₂₋₆ alkenyl,
(21) C₂₋₆ alkynyl,
(22) OH,
(23) O—C₁₋₆ alkyl,
(24) O—C₁₋₆ haloalkyl,
(25) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(26) CycA,
(27) AryA,
(28) HetA,
(29) HetR,
(30) C₁₋₆ alkyl substituted with CycA, AryA, HetA, or HetR,
(31) J-CycA,
(32) J-AryA,
(33) J-HetA, or
(34) J-HetR;

### J is:

(1) O,
(2) S,
(3) S(O),
(4) S(O)₂,
(5) O—C₁₋₆ alkylene,
(6) S—C₁₋₆ alkylene,
(7) S(O)—C₁₋₆ alkylene,
(8) S(O)₂—C₁₋₆ alkylene,
(9) N(R^{A}),
(10) N(R^{A})—C₁₋₆ alkylene,
(11) C(O),
(12) C(O)—C₁₋₆ alkylene-O,
(13) C(O)N(R^{A}),
(14) C(O)N(R^{A})—C₁₋₆ alkylene,
(15) C(O)N(R^{A})—C₁₋₆ alkylene-C(O)O, or
(16) C(O)N(R^{A})S(O)₂;
   CycA is C₃₋₈ cycloalkyl which is optionally substituted with a total of from 1 to 6 substituents, wherein:

(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and

(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
   AryA is aryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B}, or
(25) N(R^{A})CO₂R^{B}, and

(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
   HetA is heteroaryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and

(ii) from zero to 2 substituents are each independently:
(1) CycE,
(2) AryE,
(3), O-AryE,
(4) HetE,
(5) HetF, or
(6) C₁₋₆ alkyl substituted with CycE, AryE, O-AryE, HetE, or HetF;
   HetR is (i) a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, or (ii) a 6- to 10-membered saturated or mono-unsaturated, bridged or fused heterobicyclic ring containing from 1 to 4 heteroatoms independently selected from N, O and S, where each S is optionally oxidized to S(O) or S(O)₂; and wherein the saturated or mono-unsaturated heterocyclic or heterobicyclic ring is optionally substituted with a total of from 1 to 4 substituents, wherein:
(i) from zero to 4 substituents are each independently halogen, CN, C₁₋₆ alkyl, OH, oxo, C(O)R^{A}, C(O)OR^{A}, C(O)N(R^{A})R^{B}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, AryE, HetE, HetF, or C₁₋₆ alkyl substituted with CycE, AryE, HetE, or HetF;

### R² is:

(1) C₁₋₆ alkyl,
(2) C₁₋₆ haloalkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(3) CycB,
(4) AryB,
(5) HetB,
(6) HetS,
(7) C₁₋₆ alkyl substituted with CycB, AryB, HetB, or HetS,
(8) N(R^{A})—C₁₋₆ alkyl,
(9) N(R^{A})—C₁₋₆ alkyl, wherein the alkyl is substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(10) N(R^{A})-CycB,
(11) N(R^{A})-AryB,
(12) N(R^{A})—HetB, or
(13) N(R^{A})—C₁₋₆ alkyl, wherein the alkyl is substituted with CycB, AryB, HetB, or HetS;
   CycB independently has the same definition as CycA;
   AryB independently has the same definition as AryA;
   HetB independently has the same definition as HetA;
   HetS independently has the same definition as HetR;
   R³ is H or C₁₋₆ alkyl;

### R⁴ is:

(1) H,
(2) N(H)R^{A},
(3) C₁₋₆ alkyl,
(4) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) C₁₋₆ haloalkyl,
(6) C(O)—C₁₋₆ alkyl,
(7) C(O)—C₁₋₆ alkylene-O—C₁₋₆ alkyl,
(8) C(O)—C₁₋₆ alkylene-O(C═O)—C₁₋₆ alkyl,
(9) C(O)—C₁₋₆ alkylene-C(O)O—C₁₋₆ alkyl,
(10) C(O)—C₁₋₆ alkylene-N(R^{A})R^{B},
(11) C(O)—C₁₋₆ alkylene-N(R^{A})—C₂₋₆ alkylene-OH, with the proviso that the OH is not attached to the carbon in C₂₋₆ alkylene that is directly attached to the rest of the molecule,
(12) C(O)—C₁₋₆ alkylene-N(R^{A})—C₁₋₆ alkylene-N(R^{A})R^{B},
(13) C(O)—O—C₁₋₆ alkyl,
(14) C(O)N(R^{A})R^{B},
(15) C(O)N(R^{A})—C₁₋₆ alkylene-N(R^{A})R^{B},
(16) C(O)N(R^{A})—C₁₋₆ alkylene-C(O)—O—C₁₋₆ alkyl,
(17) SO₂R^{A},
(18) SO₂N(R^{A})R^{B},
(19) C₂₋₆ alkenyl,
(20) C₂₋₆ alkynyl,
(21) CycC,
(22) AryC,
(23) HetC,
(24) HetT,
(25) C₁₋₆ alkyl substituted with CycC, AryC, HetC, or HetT,
(26) C₁₋₆ alkenyl substituted with CycC, AryC, HetC, or HetT,
(27) C₁₋₆ alkynyl substituted with CycC, AryC, HetC, or HetT,
(28) L-CycC,
(29) L-AryC,
(30) L-HetC, or
(31) L-HetT;

### L is:

(1) C(O),
(2) C(O)—C₁₋₆ alkylene, wherein the C₁₋₆ alkylene is optionally substituted with from 1 to 2 substituents each of which is independently OH, C₁₋₆ haloalkyl, O—C₁₋₆ alkyl, O—C₁₋₆ haloalkyl, CN, NO₂, or N(R^{A})R^{B},
(3) C(O)—C₁₋₆ alkylene-O,
(4) C(O)—C₁₋₆ alkylene-O—C₁₋₆ alkylene,
(5) C(O)—C₁₋₆ alkylene-N(R^{A}),
(6) C(O)—C₁₋₆ alkylene-N(R^{A})—C₁₋₆ alkylene,
(7) C(O)N(R^{A}),
(8) C(O)N(R^{A})—C₁₋₆ alkylene,
(9) C(O)N(R^{A})—C₁₋₆ alkylene-C(O)O,
(10) C(O)N(R^{A})—C₁₋₆ alkylene-C(O)N(R^{A}), or
(11) S(O)₂;
   CycC independently has the same definition as CycA;
   AryC independently has the same definition as AryA;
   HetC independently has the same definition as HetA;
   HetT independently has the same definition as HetR;
   R⁵ is H or independently has the same definition as R¹;
   each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
   each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
   each CycE is independently C₃₋₈ cycloalkyl which is optionally substituted with from 1 to 4 substituents each of which is independently halogen, C₁₋₆ alkyl, OH, O—C₁₋₆ alkyl, C₁₋₆ haloalkyl, or O—C₁₋₆ haloalkyl;
   each AryE is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
   each HetE is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, N(R^{A})R^{B}, N(R^{A})C(O)N(R^{A})R^{B}, or N(R^{A})CO₂R^{B};
   each HetF is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with a total of from 1 to 4 substituents, each of which is independently halogen, CN, C₁₋₆ alkyl, OH, oxo, O—C₁₋₆ alkyl, C₁₋₆ haloalkyl, or O—C₁₋₆ haloalkyl;
   each R^{A} is independently H or C₁₋₆ alkyl; and
   each R^{B} is independently H or C₁₋₆ alkyl;
   and with the proviso that:

(A) when R¹ is chloro, R² is AryB and AryB is unsubstituted phenyl or 4-methylphenyl, R³ is H, and R⁵ is H, then R⁴ is not unsubstituted phenyl, and

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂.

Other embodiments, aspects and features of the present invention are either further described in or will be apparent from the ensuing description, examples and appended claims.

### DETAILED DESCRIPTION OF THE INVENTION

The compounds of Formula I above, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. Based upon the testing of representative compounds of the invention in the assay set forth in Example 98 below, it is known that compounds of Formula I inhibit the RNA-dependent DNA polymerase activity of HIV-1 reverse transcriptase. The compounds can also exhibit activity against drug resistant forms of HIV (e.g., mutant strains of HIV in which reverse transcriptase has a mutation at lysine 103→asparagine (K103N) and/or tyrosine 181→cysteine (Y181C)), and thus can exhibit decreased cross-resistance against currently approved antiviral therapies.

A first embodiment of the present invention is a compound of Formula I, or a pharmaceutically acceptable salt thereof, wherein each of the variables is as originally defined above (i.e., as defined in the Summary of the Invention); and with the proviso that:

(A) when R¹ is halogen, R² is AryB and AryB is unsubstituted phenyl or phenyl substituted with C₁₋₆ alkyl, R³ is H, and R⁵ is H, then R⁴ is not unsubstituted phenyl, and

(B) when R³ is H, then R⁴ is not NH₂.

A second embodiment of the present invention is a compound of Formula I, or a pharmaceutically acceptable salt thereof, wherein each of the variables is as originally defined above; and with the proviso that:

(A) when R¹ is chloro, R² is AryB and AryB is unsubstituted phenyl or 4-methylphenyl, R³ is H, and R⁵ is H, then R⁴ is not unsubstituted phenyl,

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂,

and including any one or more of the following provisos:

(C) when R² is AryB, then AryB is not a phenyl that is di-substituted or trisubstituted with OCH₃, or

(D) when R⁵ is attached to the 6-position of the indole ring and is O—C₁₋₆ alkyl (e.g., methoxy), then R¹ is not oxazol-5-yl,

(E) when R¹ is CH₂-AryA or J-AryA, J in the definition of R¹ is O, S, S(O), S(O)₂, NH, or N(C₁₋₄ alkyl), and R⁵ is H, OH, halogen, CN, NO₂, C₁₋₄ alkyl, N(R^{A})R^{B}, N(R^{A})-CycA, N(R^{A})—CH₂-phenyl, or N(R^{A})-phenyl, wherein either of the phenyl groups is optionally substituted with a total of from 1 to 5 substituents wherein (i) from zero to 5 substituents are each independently halogen, OH, NH₂, CO₂H, O—C₁₋₄ alkyl, C(O)O—C₁₋₄ alkyl, NHC(O)O—C₁₋₄ alkyl, and (ii) from zero to 2 substituents are each independently HetE, HetF, or phenyl optionally substituted by halogen or OH, then AryA in the definition of R¹ is not a di- or tri-substituted phenyl in which (i) one substituent in the di-substituted phenyl or each of two substituents in the ti-substituted phenyl is independently halogen, CN, C₁₋₆ alkyl, CF₃, CHF₂, CH₂F, or C₃₋₇ cycloalkyl, wherein either the one substituent on the di-substituted phenyl or one or both of the two substituents in the tri-substituted phenyl is ortho to the CH₂ or J moiety linking AryA to the rest of the molecule and (ii) the other substituent in the di- or tri-substituted phenyl is OC(O)N(R^{A})R^{B}, S(O)₂R^{A}, S(O)₂N(R^{A})R^{B}, N(R^{A})S(O)₂R^{B}, N(R^{A})S(O)₂N(R^{A})R^{B}, N(R^{A})C(O)R^{B}, N(R^{A})C(O)N(R^{A})R^{B}, N(R^{A})CO₂R^{B}, HetE, HetF, (CH₂)₁₋₂-HetE, or (CH₂)₁₋₂-HetF,

(F) when R¹ is CH₂CH₂-HetA or J-HetA, J in the definition of R¹ is OCH₂, SCH₂, or S(O)₂CH₂, and HetA in the definition of R¹ is (i) a 5- or 6-membered heteroaromatic ring containing from 1 to 3 N atoms wherein the ring is optionally mono- or di-substituted, (ii) a 5-membered heteroaromatic ring containing one O or S atom and from zero to 2 N atoms, wherein the ring is optionally mono- or di-substituted, or (iii) a 9- or 10-membered aromatic bicyclic, fused ring system containing from 1 to 3 N atoms, wherein the ring system is optionally mono- or di-substituted, then R⁴ is not SO₂R^{A} or C₁₋₆ alkyl substituted with OH, C(O)N(R^{A})R^{B}, CO₂R^{A}, SO₂N(R^{A})R^{B}, or N(R^{A})SO₂R^{B}, and

(G) when R¹ is CH₂CH₂-AryA or J-AryA, J in the definition of R¹ is OCH₂, SCH₂, or S(O)₂CH₂, and AryA in the definition of R¹ is (i) an aryl other than phenyl, wherein the aryl other than phenyl is optionally mono- or di-substituted, then R⁴ is not SO₂R^{A} or C₁₋₆ alkyl substituted with OH, C(O)N(R^{A})R^{B}, CO₂R^{A}, SO₂N(R^{A})R^{B}, or N(R^{A})SO₂R^{B}.

A third embodiment of the present invention is a compound of Formula I, or a pharmaceutically acceptable salt thereof, wherein each of the variables is as originally defined above; and with the proviso that:

(A) when R¹ is halogen, R² is AryB and AryB is unsubstituted phenyl or phenyl substituted with C₁₋₆ alkyl, R³ is H, and R⁵ is H, then R⁴ is not unsubstituted phenyl,

(B) when R³ is H, then R⁴ is not NH₂,

and including any one or more of the following provisos:

(C) (i) when R² is AryB, then AryB is not an aryl that is di-substituted or tri-substituted with O—C₁₋₆ alkyl or (ii) when R² is HetB, then HetB is not a heteroaryl that is di-substituted or trisubstituted with O—C₁₋₆ alkyl,

(D) when R⁵ is attached to the 6-position of the indole ring and is other than H, then R¹ is not unsubstituted oxazolyl or substituted oxazolyl,

(E) when R¹ is C₁₋₆ alkylene-AryA or J-AryA, J in the definition of R¹ is O, S, S(O), S(O)₂, or N(R^{A}), then AryA is not a di- or tri-substituted phenyl in which at least one of the substituents in the di- or tri-substituted phenyl is ortho to the C₁₋₆ alkylene or J moiety linking AryA to the rest of the molecule,

(F) when HetA is (i) a 5- or 6-membered heteroaromatic ring containing from 1 to 3 N atoms wherein the ring is optionally mono- or di-substituted, (ii) a 5-membered heteroaromatic ring containing one O or S atom and from zero to 2 N atoms, wherein the ring is optionally mono- or di-substituted, or (iii) a 9- or 10-membered aromatic bicyclic, fused ring system containing from 1 to 3 N atoms, wherein the ring system is optionally mono- or di-substituted, then R¹ is not C₁₋₆ alkyl substituted with HetA or J-HetA, and

(G) when AryA is an aryl other than phenyl, wherein the aryl other than phenyl is optionally mono- or di-substituted, then R¹ is not C₁₋₆ alkylene-AryA or J-AryA.

A fourth embodiment of the present invention is a compound of Formula I, or a pharmaceutically acceptable salt thereof, wherein R¹ is:
(1) halogen,
(2) CN,
(3) NO₂,
(4) N(R^{A})R^{B},
(5) N(R^{A})S(O)₂R^{B},
(6) N(R^{A})C(O)R^{B},
(7) C₁₋₆ alkyl,
(8) C₁₋₆ haloalkyl,
(9) C₂₋₆ alkenyl,
(10) OH,
(11) O—C₁₋₆ alkyl,
(12) O—C₁₋₆ haloalkyl,
(13) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(14) CycA,
(15) AryA,
(16) HetA, or
(17) C₁₋₆ alkyl substituted with CycA, AryA, or HetA; and

### R⁵ is H;

and all other variables are as originally defined; and with the proviso that:

(A) when R¹ is chloro, R² is AryB and AryB is unsubstituted phenyl or 4-methylphenyl, and R³ is H, then R⁴ is not unsubstituted phenyl, and

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H or (ii) when R³ is H and R⁵ is other than H, then R⁴ is not NH₂.

A first aspect of the fourth embodiment is a compound of Formula I, or a pharmaceutically acceptable salt thereof, wherein the compound is as defined in the fourth embodiment, except that it incorporates the provisos set forth in the first embodiment. A second aspect of the fourth embodiment is a compound of Formula I, or a pharmaceutically acceptable salt thereof, wherein the compound is as defined in the fourth embodiment, except that it incorporates provisos A, B and any one or more of provisos C and E to G as set forth in the second embodiment. A third aspect of the fourth embodiment is a compound of Formula I, or a pharmaceutically acceptable salt thereof, wherein the compound is as defined in the fourth embodiment, except that it incorporates provisos A, B and any one or more of provisos C and E to G as set forth in the third embodiment. It is understood that the provisos set forth in the foregoing aspects of the fourth embodiment can be modified to conform with the definitions of the variables set forth in the fourth embodiment. For example, since J-HetA and J-AryA are not included in the definition of R¹ in the fourth embodiment, provisos E, F and G in the second and third aspects can be modified to remove the language directed to J-HetA and J-AryA. It is also noted that, since R⁵ is H in the fourth embodiment, proviso D does not restrict the scope of the fourth embodiment and thus is not included in the second or third aspect of the fourth embodiment.

A fifth embodiment of the present invention is a compound of Formula I, or a pharmaceutically acceptable salt thereof, wherein R² is: (1) AryB, (2) HetB, (3) HetS, (4) C₁₋₆ alkyl substituted with AryB or HetB, (5) N(R^{A})-AryB, or (6) N(R^{A})-HetB; and all other variables are as originally defined; and with the proviso that:

(A) when R¹ is chloro, R² is AryB and AsyB is unsubstituted phenyl or 4-methylphenyl, and R³ is H, then R⁴ is not unsubstituted phenyl, and

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H or (ii) when R³ is H and R⁵ is other than H, then R⁴ is not NH₂.

An aspect of the fifth embodiment is a compound of Formula I, or a pharmaceutically acceptable salt thereof, wherein the compound is as defined in the fifth embodiment, except that it incorporates the provisos set forth in the first embodiment. Another aspect of the fifth embodiment is a compound of Formula I, or a pharmaceutically acceptable salt thereof, wherein the compound is as defined in the fifth embodiment, except that it incorporates the provisos set forth in the second embodiment. Still another aspect of the fifth embodiment is a compound of Formula I, or a pharmaceutically acceptable salt thereof, wherein the compound is as defined in the fifth embodiment, except that it incorporates the provisos set forth in the third embodiment.

A sixth embodiment of the present invention is a compound of Formula I, or a pharmaceutically acceptable salt thereof, wherein R³ is H; and all other variables are as originally defined; and with the proviso that:

(A) when R¹ is chloro, R² is AryB and AryB is unsubstituted phenyl or 4-methylphenyl, and R⁵ is H, then R⁴ is not unsubstituted phenyl, and

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, and R⁵ is H or (ii) when R⁵ is other than H, then R⁴ is not NH₂.

Aspects of the sixth embodiment include a compound of Formula I, or a pharmaceutically acceptable salt thereof, as defined in the sixth embodiment incorporating the provisos as set forth in any one of the first, second and third embodiments.

A seventh embodiment of the present invention is a compound of Formula I, or a pharmaceutically acceptable salt thereof, wherein R⁴ is:
(1) C₁₋₆ alkyl,
(2) C₁₋₆ alkyl substituted with O—C₁₋₆ alkyl, O—C₁₋₆ haloalkyl, N(R^{A})R^{B}, C(O)N(R^{A})R^{B}, C(O)R^{A}, CO₂R^{A}, or OC(O)N(R^{A})R^{B},
(3) C₁₋₆ haloalkyl,
(4) C(O)—C₁₋₆ alkyl,
(5) C(O)—C₁₋₆ alkylene-O—C₁₋₆ alkyl,
(6) C(O)—C₁₋₆ alkylene-O(C═O)—C₁₋₆ alkyl,
(7) C(O)—C₁₋₆ alkylene-C(O)O—C₁₋₆ alkyl,
(8) C(O)—C₁₋₆ alkylene-N(R^{A})R^{B},
(9) C(O)—C₁₋₆ alkylene-N(R^{A})—C₂₋₆ alkylene-OH, with the proviso that the OH is not attached to the carbon in C₂₋₆ alkylene that is directly attached to the rest of the molecule,
(10) C(O)—C₁₋₆ allylene-N(R^{A})—C₁₋₆ alkylene-N(R^{A})R^{B},
(11) C(O)N(R^{A})R^{B},
(12) C(O)N(R^{A})—C₁₋₆ alkylene-N(R^{A})R^{B},
(13) C(O)N(R^{A})—C₁₋₆ alkylene-C(O)—O—C₁₋₆ alkyl,
(14) CycC,
(15) AryC,
(16) HetC,
(17) HetT,
(18) C₁₋₆ alkyl substituted with CycC, AryC, HetC, or HetT
(19) L-CycC,
(20) L-AryC,
(21) L-HetC, or
(22) L-HetT; and

### L is:

(1) C(O),
(2) C(O)—C₁₋₆ alkylene, wherein the C₁₋₆ alkylene is optionally substituted with from 1 to 2 substituents each of which is independently OH, C₁₋₆ haloalkyl, O—C₁₋₆ alkyl, or O—C₁₋₆ haloalkyl,
(3) C(O)—C₁₋₆ alkylene-O,
(4) C(O)—C₁₋₆ allylene-O—C₁₋₆ alkylene,
(5) C(O)—C₁₋₆ alkylene-N(R^{A}),
(6) C(O)—C₁₋₆ allylene-N(R^{A})—C₁₋₆ alkylene,
(7) C(O)N(R^{A}), or
(8) C(O)N(R^{A})—C₁₋₆ alkylene;
   and all other variables are as originally defined; and with the proviso that:

(A) when R¹ is chloro, R² is AryB and AryB is unsubstituted phenyl or 4-methylphenyl, R³ is H, and R⁵ is H, then R⁴ is not unsubstituted phenyl.

Aspects of the seventh embodiment include a compound of Formula I, or a pharmaceutically acceptable salt thereof, as defined in the seventh embodiment incorporating proviso A of the first embodiment, or proviso A and any one or more of provisos C to G of the second embodiment, or proviso A and any one or more of provisos C to G of the third embodiment. It is understood that the provisos set forth in the foregoing aspects of the seventh embodiment can be modified to conform with the definitions of the variables set forth in the seventh embodiment. For example, the restrictions placed on R⁴ in provisos F and G can be modified to conform with the definition of R⁴ in the seventh embodiment.

An eighth embodiment of the present invention is a compound of Formula I, or a pharmaceutically acceptable salt thereof, wherein HetS is a 4- to 7-membered, saturated or mono-unsaturated heterocyclic ring or a 6- to 10-membered, saturated or mono-unsaturated, fused or bridged heterobicyclic ring, wherein the heterocyclic or heterobicyclic ring contains a nitrogen atom which is directly attached to the rest of the molecule and optionally contains an additional heteroatom selected from N, O, and S, where the S is optionally oxidized to S(O) or S(O)₂; and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, C(O)R^{A}, C(O)OR^{A}, C(O)N(R^{A})R^{B}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, HetE, AryE, or C₁₋₆ allyl substituted with CycE, AryE, HetE, or HetF;
   and all other variables are as originally defined; and with the proviso that:

(A) when R¹ is chloro, R² is AryB and AryB is unsubstituted phenyl or 4-methylphenyl, R³ is H, and R⁵ is H, then R⁴ is not unsubstituted phenyl, and

(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂.

Aspects of the eighth embodiment include a compound of Formula I, or a pharmaceutically acceptable salt thereof, as defined in the eighth embodiment incorporating the provisos as set forth in any one of the first, second and third embodiments.

A ninth embodiment of the present invention is a compound of Formula I, or a pharmaceutically acceptable salt thereof, wherein each R^{A} and R^{B} is independently —H or —C₁₋₄ alkyl; and all other variables are as originally defined or as defined in any one of the preceding embodiments or aspects thereof.

A tenth embodiment of the present invention is a compound of Formula I, or a pharmaceutically acceptable salt thereof, wherein each R^{A} and R^{B} is independently —H or methyl; and all other variables are as originally defined or as defined in any one of the preceding embodiments or aspects thereof.

A first class of the present invention includes compounds of Formula I and pharmaceutically acceptable salts thereof, wherein:

### R¹ is:

(1) Cl, Br, or F,
(2) CN,
(3) NO₂,
(4) N(H)—C₁₋₄ alkyl,
(5) N(C₁₋₄ alkyl)₂,
(6) N(H)S(O)₂—C₁₋₄ alkyl,
(7) N(C₁₋₄ alkyl)S(O)₂—C₁₋₄ alkyl,
(8) N(H)C(O)—C₁₋₄ alkyl,
(9) N(C₁₋₄ allyl)C(O)—C₁₋₄ alkyl,
(10) C₁₋₄ allyl,
(11) C₁₋₄ haloalkyl,
(12) CH═CH₂,
(13) OH,
(14) O—C₁₋₄ alkyl,
(15) O—C₁₋₄ haloalkyl,
(16) C₁₋₄ alkyl substituted with OH, O—C₁₋₄ alkyl, CN, NO₂, N(H)—C₁₋₄ alkyl, or N(C₁₋₄ alkyl)₂,
(17) CycA,
(18) AryA,
(19) HetA, or
(20) C₁₋₄ alkyl substituted with CycA, AryA, or HetA;

### R² is

(1) C₁₋₄ alkyl,
(2) C₁₋₄ haloalkyl,
(3) C₁₋₄ alkyl substituted with OH, O—C₁₋₄ alkyl, O—C₁₋₄ fluoroalkyl, CN, NO₂, N(H)—C₁₋₄ alkyl, or N(C₁₋₄ alkyl)₂,
(4) CycB,
(5) AryB,
(6) HetB,
(7) HetS,
(8) C₁₋₄ alkyl substituted with CycB, AryB, HetB, or HetS,
(9) N(H)—C₁₋₄ alkyl,
(10) N(H)—C₁₋₄ alkyl, wherein the C₁₋₄ alkyl is substituted with OH, O—C₁₋₄ alkyl, O—C₁₋₄ fluoroalkyl, CN, NO₂, N(H)—C₁₋₄ alkyl, or N(C₁₋₄ alkyl)₂, with the proviso that the OH, O—C₁₋₄ alkyl, or O—C₁₋₄ fluoroalkyl is not attached to the carbon in C₁₋₄ alkyl that is directly attached to the rest of the molecule,
(11) N(H)-CycB,
(12) N(H)-AryB,
(13) N(H)-HetB, or
(14) N(H)—C₁₋₆ alkyl, wherein the alkyl is substituted with CycB, AryB, HetB, or HetS;

### R³ is H;

### R⁴ is:

(1) C(O)—C₁₋₄ alkyl,
(2) C(O)—(CH₂)₁₋₄—O—C₁₋₄ alkyl,
(3) C(O)—(CH₂)₁₋₄—O(C═O)—C₁₋₄ alkyl,
(4) C(O)—(CH₂)₁₋₄—C(O)O—C₁₋₄ alkyl,
(5) C(O)—(CH₂)₁₋₄—N(H)—C₁₋₄ alkyl,
(6) C(O)—(CH₂)₁₋₄—N(C₁₋₄ alkyl)₂,
(7) C(O)—(CH₂)₁₋₄—N(H)—(CH₂)₂₋₅OH,
(8) C(O)—(CH₂)₁₋₄—N(H)—(CH₂)₁₋₄—N(H)—C₁₋₄ alkyl,
(9) C(O)—(CH₂)₁₋₄—N(H)—(CH₂)₁₋₄—N(C₁₋₄ alkyl)₂,
(10) C(O)N(H)—C₁₋₆ alkyl,
(11) C(O)N(C₁₋₄ alkyl)₂,
(12) C(O)N(H)—(CH₂)₁₋₄—N(H)—C₁₋₄ alkyl,
(13) C(O)N(H)—(CH₂)₁₋₄—N(C₁₋₄ alkyl)₂,
(14) C(O)N(H)—(CH₂)₁₋₄—C(O)—O—C₁₋₄ alkyl,
(15) CycC,
(16) AryC,
(17) HetC,
(18) HetT,
(19) CH(CH₃)-CycC, CH(CH₃)-AryC, CH(CH₃)-HetC, or CH(CH₃)-HetT
(20) (CH₂)₁₋₄-CycC, (CH₂)₁₋₄-AryC, (CH₂)₁₋₄-HetC, or (CH₂)₁₋₄-HetT
(21) L-CycC,
(22) L-AryC,
(23) L-HetC, or
(24) L-HetT; and

### L is:

(1) C(O),
(2) C(O)—(CH₂)₁₋₄, wherein the (CH₂)₁₋₄ is optionally substituted with from 1 to 2 substituents each of which is independently OH, CF₃, O—C₁₋₄ alkyl, or OCF₃,
(3) C(O)—(CH₂)₁₋₄—O,
(4) C(O)—(CH₂)₁₋₄—O—(CH₂)₁₋₄,
(5) C(O)—(CH₂)₁₋₄—O—CH(CH₃),
(6) C(O)—(CH₂)₁₋₄—N(H),
(7) C(O)—(CH₂)₁₋₄—N(C₁₋₄ amyl),
(8) C(O)—(CH₂)₁₋₄—N(H)—(CH₂)₁₋₄,
(9) C(O)—(CH₂)₁₋₄—N(C₁₋₄ alkyl)-(CH₂)₁₋₄,
(10) C(O)—(CH₂)₁₋₄—N(H)—CH(CH₃),
(11) C(O)—(CH₂)₁₋₄—N(C₁₋₄ alkyl)-CH(CH₃),
(12) C(O)N(H),
(13) C(O)N(C₁₋₄ alkyl),
(14) C(O)N(H)—(CH₂)₁₋₄, or
(15) C(O)N(C₁₋₄ alkyl)-(CH₂)₁₋₄;

### R⁵ is H;

CycA is C₃₋₆ cycloalkyl which is optionally substituted with a total of from 1 to 4 substituents, wherein:

(i) from zero to 4 substituents are each independently:
(1) Cl, Br, or F,
(2) CN,
(3) C₁₋₄ allyl,
(4) OH,
(5) O—C₁₋₄ alkyl, or
(6) C₁₋₄ haloalkyl, and

(ii) from zero to 1 substituent is AryE, HetE, CH₂-AryE, or CH₂-HetE;

AryA is phenyl or naphthyl, wherein the phenyl or naphthyl is optionally substituted with a total of from 1 to 5 substituents, wherein:

(i) from zero to 5 substituents are each independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) halogen,
(7) CN,
(8) NO₂,
(9) NH₂,
(10) N(H)—C₁₋₄ alkyl,
(11) N(C₁₋₄ alkyl)₂,
(12) C(O)NH₂,
(13) C(O)N(H)—C₁₋₄ alkyl,
(14) C(O)N(C₁₋₄ alkyl)₂,
(15) C(O)—C₁₋₄ alkyl,
(16) CO₂—C₁₋₄ alkyl,
(17) S—C₁₋₄ allyl,
(18) S(O)—C₁₋₄ alkyl,
(19) SO₂—C₁₋₄ alkyl,
(20) SO₂NH₂,
(21) SO₂N(H)—C₁₋₄ alkyl,
(22) SO₂N(C₁₋₄ alkyl)₂,
(23) SO₂N(H)C(O)—C₁₋₄ alkyl,
(24) SO₂N(C₁₋₄ alkyl)C(O)—C₁₋₄ alkyl,
(25) N(H)C(O)—C₁₋₄ alkyl, or
(26) N(C₁₋₄ alkyl)C(O)—C₁₋₄ alkyl, and

(ii) from zero to 1 substituent is AryE, HetE, CH₂-AryE, or CH₂-HetE;

HetA is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing a total of from 1 to 4 heteroatoms independently selected from zero to 4 N atoms, zero to 2 O atoms, and zero to 2 S atoms, wherein either one or both of the rings contain one or more of the heteroatoms, at least one ring is aromatic, each N is optionally in the form of an oxide, and each S in a ring which is not aromatic is optionally S(O) or S(O)₂; wherein the heteroaromatic ring or the bicyclic, fused ring system is optionally substituted with a total of from 1 to 4 substituents, wherein:

(i) from zero to 4 substituents are each independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) Cl, Br, or F,
(7) CN,
(8) C(O)N(H)—C₁₋₄ alkyl,
(9) C(O)N(C₁₋₄ alkyl)₂,
(10) S(O)₂—C₁₋₄ alkyl,
(11) S(O)₂NH₂,
(12) S(O)₂N(H)—C₁₋₄ alkyl, or
(13) S(O)₂N(C₁₋₄ allyl)₂, and

(ii) from zero to 1 substituent is AryE, HetE, CH₂-AryE, or CH₂-HetE;

CycB and CycC each independently have the same definition as CycA;
AryB and AryC each independently have the same definition as AryA;
HetB and HetC each independently have the same definition as HetA;
HetS is a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring contains a nitrogen atom which is directly attached to the rest of the molecule and optionally contains an additional heteroatom selected from N, O, and S, where the S is optionally oxidized to S(O) or S(O)₂; and wherein the heterocyclic or heterobicyclic ring is optionally substituted with a total of from 1 to 4 substituents, wherein:
(i) from zero to 4 substituents are each independently Cl, Br, F, C₁₋₄ alkyl, OH, oxo, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
   HetT is a 4- to 7-membered, saturated or mono-unsaturated heterocyclic ring containing from 1 or 2 heteroatoms independently selected from N, O, and S, where each S is optionally oxidized to S(O) or S(O)₂, and wherein the saturated or mono-unsaturated heterocyclic ring is optionally substituted with a total of from 1 to 4 substituents, wherein:
(i) from zero to 4 substituents are each independently Cl, Br, F, C₁₋₄ alkyl, OH, oxo, C(O)NH₂, C(O)N(H)—C₁₋₄ alkyl, C(O)N(C₁₋₄ alkyl)₂, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
   AryE is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, Cl, Br, or F, CN, C(O)N(H)—C₁₋₄ alkyl, C(O)N(C₁₋₄ alkyl)₂, S(O)₂—C₁₋₄ alkyl, S(O)₂NH₂, S(O)₂N(H)—C₁₋₄ alkyl, or S(O)₂N(C₁₋₄ alkyl)₂; and
   HetE is a 5- or 6-membered heteroaromatic ring containing from 1 to 4 heteroatoms independently selected from N, O and S, wherein each N is optionally in the form of an oxide, wherein the heteroaromatic ring is optionally substituted with from 1 to 3 substituents each of which is independently Cl, Br, F, CN, NO₂, C₁₋₄ alkyl, C₁₋₄ fluoroalkyl, OH, O—C₁₋₄ alkyl, or O—C₁₋₄ fluoroalkyl.
   and with the proviso that:

(A) when R¹ is Cl, and R² is AryB and AryB is unsubstituted phenyl or 4-methylphenyl, then R⁴ is not unsubstituted phenyl.

A first sub-class of the first class includes compounds of Formula I and pharmaceutically acceptable salts thereof, wherein all of the variables are as originally defined in the first class; and with the proviso that: (A) when R¹ is Cl, Br, or F, and R² is AryB and AryB is unsubstituted phenyl or phenyl substituted with C₁₋₄ alkyl, then R⁴ is not unsubstituted phenyl.

A second sub-class of the first class includes compounds of Formula I and pharmaceutically acceptable salts thereof, wherein all of the variables are as originally defined in the first class; and with the proviso that:

(A) when R¹ is Cl, and R² is AryB and AryB is unsubstituted phenyl or 4-methylphenyl, then R⁴ is not unsubstituted phenyl,

and including either or both of the following provisos:

(C) when R² is AryB, then AryB is not a phenyl that is di-substituted or trisubstituted with OCH₃, and

(E) when R¹ is CH₂-AryA, then AryA in the definition of R¹ is not a di- or tri-substituted phenyl in which (i) one substituent in the di-substituted phenyl or each of two substituents in the tri-substituted phenyl is independently halogen, CN, C₁₋₄ alkyl, CF₃, CHF₂, or CH₂F, wherein either the one substituent on the di-substituted phenyl or one or both of the two substituents in the tri-substituted phenyl is ortho to the CH₂ moiety linking AryA to the rest of the molecule and (ii) the other substituent in the di- or tri-substituted phenyl is S(O)₂—C₁₋₄ alkyl, SO₂NH₂, SO₂N(H)—C₁₋₄ alkyl, SO₂N(C₁₋₄ alkyl)₂, N(H)C(O)—C₁₋₄ alkyl, N(C₁₋₄ alkyl)C(O)—C₁₋₄ alkyl, HetE, or CH₂-HetE.

A third sub-class of the first class is identical to the second sub-class, except that proviso A is as follows: when R¹ is Cl, Br, or F, and R² is AryB and AryB is unsubstituted phenyl or phenyl substituted with C₁₋₄ alkyl, then R⁴ is not unsubstituted phenyl.

A fourth sub-class of the first class includes compounds of Formula I and pharmaceutically acceptable salts thereof, wherein all of the variables are as originally defined in the first class; and with the proviso that:

(A) when R¹ is Cl, Br, or F, and R² is AryB and AryB is unsubstituted phenyl o phenyl substituted with C₁₋₄ alkyl, then R⁴ is not unsubstituted phenyl,

and including any one or more of the following proviso:

(C) (i) when R² is AryB, then AryB is not an aryl that is di-substituted or tri-substituted with O—C₁₋₄ allyl or (ii) when R² is HetB, then HetB is not a heteroaryl that is di-substituted or trisubstituted with O—C₁₋₄ alkyl,

(E) when R¹ is C₁₋₄ alkyl substituted with AryA, then AryA in the definition of R¹ is not a di- or tri-substituted phenyl in which at least one of the substituents in the di- or tri-substituted phenyl is ortho to the C₁₋₆ alkylene,

(F) when HetA in the definition of R¹ is (i) a 5- or 6-membered heteroaromatic ring containing from 1 to 3 N atoms wherein the ring is optionally mono- or di-substituted, (ii) a 5-membered heteroaromatic ring containing one O or S atom and from zero to 2 N atoms, wherein the ring is optionally mono- or di-substituted, or (iii) a 9- or 10-membered aromatic bicyclic, fused ring system containing from 1 to 3 N atoms, wherein the ring system is optionally mono- or di-substituted, then R¹ is not C₁₋₄ alkyl substituted with HetA, and

(G) when AryA in the definition of R¹ is naphthyl which is optionally mono- or di-substituted, then R¹ is not C₁₋₄ alkylene-AryA.

A second class of the present invention includes compounds of Formula I and pharmaceutically acceptable salts thereof, wherein:

R¹ is chlorine or bromine;

### R² is AryB or HetS;

AryB is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, C(O)N(H)—C₁₋₄ alkyl, C(O)N(C₁₋₄ alkyl)₂, S(O)₂—C₁₋₄ alkyl, S(O)₂NH₂, S(O)₂N(H)—C₁₋₄ alkyl, or S(O)₂N(C₁₋₄ alkyl)₂;
HetS is a saturated heterocyclic or heterobicyclic ring selected from the group consisting of:

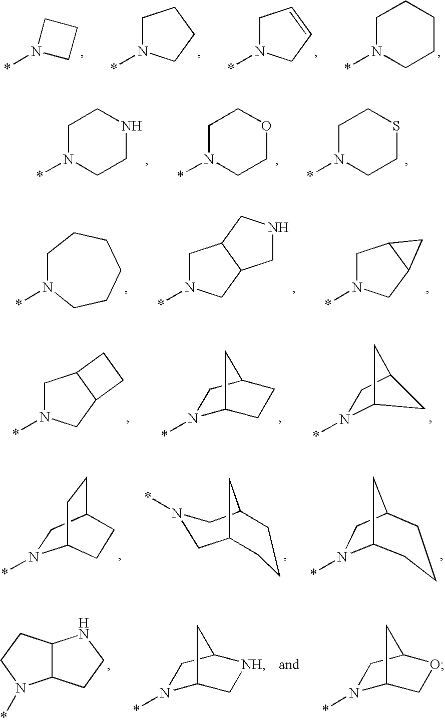

wherein the asterisk denotes the point of attachment of the heterocyclic or heterobicyclic ring to the rest of the molecule, and wherein the heterocyclic or heterobicyclic ring is optionally substituted with a total of from 1 to 4 substituents, each of which is independently C₁₋₄ alkyl, S(O)₂—C₁₋₄ alkyl, O—C₁₋₄, alkyl, C₁₋₄ fluoroalkyl, O—C₁₋₄ fluoroalkyl, oxo, Cl, Br, or F;

### R³ is H;

### R⁴ is:

(1) C(O)—C₁₋₄ alkyl,
(2) C(O)—(CH₂)₁₋₃—O—C₁₋₄ alkyl,
(3) C(O)—(CH₂)₁₋₃—O(C═O)—C₁₋₄ alkyl,
(4) C(O)—(CH₂)₁₋₃—C(O)O—C₁₋₄ alkyl,
(5) C(O)—(CH₂)₁₋₃—N(H)—C₁₋₄ alkyl,
(6) C(O)—(CH₂)₁₋₃—N(C₁₋₄ alkyl)₂,
(7) C(O)—(CH₂)₁₋₃—N(H)—(CH₂)₂₋₅OH,
(8) C(O)—(CH₂)₁₋₃—N(H)—(CH₂)₁₋₃—N(H)—C₁₋₄ alkyl,
(9) C(O)—(CH₂)₁₋₃—N(H)—(CH₂)₁₋₃—N(C₁₋₄ alkyl)₂,
(10) C(O)NH₂,
(11) C(O)N(H)—C₁₋₄ alkyl,
(12) C(O)N(H)—(CH₂)₂—C₃₋₄ alkyl,
(13) C(O)N(H)—CH₂—C₄ alkyl,
(14) C(O)N(C₁₋₄ alkyl)₂,
(15) C(O)N(H)—(CH₂)₁₋₃—N(H)—C₁₋₄ alkyl,
(16) C(O)N(H)—(CH₂)₁₋₃—N(C₁₋₄ alkyl)₂,
(17) C(O)N(H)—(CH₂)₁₋₃—C(O)—O—C₁₋₄ alkyl,
(18) L-CycC,
(19) L-AryC,
(20) L-HetC, or
(21) L-HetT; and

### L is:

(1) C(O),
(2) C(O)—(CH₂)₁₋₃, wherein the (CH₂)₁₋₃ is optionally substituted with from 1 to 2 substituents each of which is independently OH, CF₃, O—C₁₋₄ alkyl, or OCF₃,
(3) C(O)—(CH₂)₁₋₃—O,
(4) C(O)—(CH₂)₁₋₃—O—(CH₂)₁₋₃,
(5) C(O)—(CH₂)₁₋₃—O—CH(CH₃),
(6) C(O)—(CH₂)₁₋₃—N(H),
(7) C(O)—(CH₂)₁₋₃—N(C₁₋₄ alkyl),
(8) C(O)—(CH₂)₁₋₃—N(H)—(CH₂)₁₋₃,
(9) C(O)—(CH₂)₁₋₃—N(C₁₋₄ alkyl)-(CH₂)₁₋₃,
(10) C(O)—(CH₂)₁₋₃—N(H)—CH(CH₃),
(11) C(O)—(CH₂)₁₋₃—N(C₁₋₄ alkyl)-CH(CH₃),
(12) C(O)N(H),
(13) C(O)N(C₁₋₄ alkyl),
(14) C(O)N(H)—(CH₂)₁₋₃, or
(15) C(O)N(C₁₋₄ alkyl)-(CH)₁₋₃;
   CycC is C₃₋₆ cycloalkyl which is optionally substituted with phenyl;
   AryC independently has the same definition as AryB;
   HetC is (i) a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl or (ii) a bicyclic, fused ring system selected from the group consisting of quinolinyl, isoquinolinyl, quinazolinyl, naphthyridinyl, benzoxazinyl, cinnolinyl, benzofuranyl, 2,3-dihydrobenzo-1,4-dioxinyl, and benzo-1,3-dioxolyl; wherein the heteroaromatic ring or the bicyclic, fused ring system is optionally substituted with a total of from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, or F;
   HetT is a saturated or mono-unsaturated heterocyclic ring selected from the group consisting of

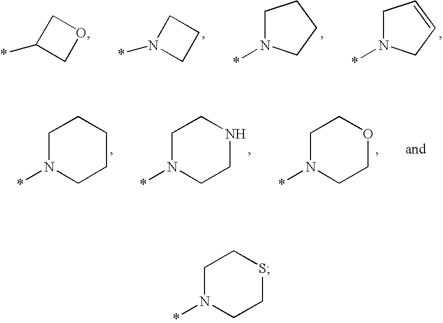

wherein the asterisk denotes the point of attachment of the heterocyclic ring to the rest of the molecule, and wherein the saturated or mono-unsaturated heterocyclic ring is optionally substituted with a total of from 1 to 4 substituents, wherein
(i) from zero to 4 substituents are each independently C₁₋₄ alkyl, C(O)NH₂, C(O)N(H)—C₁₋₄ alkyl, C(O)N(C₁₋₄ alkyl)₂, S(O)₂—C₁₋₄ alkyl, O—C₁₋₄ alkyl, C₁₋₄ fluoroalkyl, O—C₁₋₄ fluoroalkyl, oxo, Cl, Br, or F, and
(ii) from zero to 1 substituent is AryE, HetE, CH₂-AryE, or CH₂-HetE;
   AryE is phenyl which is optionally substituted with from 1 to 3 substituents each of which is independently C₁₋₄ alkyl, O—C₁₋₄ alkyl, CF₃, OCF₃, Cl, Br, or F; and
   HetE is pyridinyl which is optionally substituted with from 1 to 3 substituents each of which is independently Cl, Br, F, CN, NO₂, C₁₋₄ alkyl, CF₃, OH, O—C₁₋₄ alkyl, or OCF₃.

A first sub-class of the second class includes compounds of Formula I and pharmaceutically acceptable salts thereof, wherein all of the variables are as originally defined in the second class; and with the proviso that: (C) when (i) R² is AryB, then AryB is not an aryl that is di-substituted or trisubstituted with O—C₁₋₄ alkyl.

A third class of the present invention includes compounds of Formula I and pharmaceutically acceptable salts thereof, wherein R¹ is chlorine; and R² is AryB; and AryB is phenyl which is optionally substituted with from 1 to 3 substituents each of which is independently C₁₋₄ alkyl, O—C₁₋₄ alkyl, CF₃, OCF₃, OH, Cl, Br, F, CN, C(O)N(H)CH₃, C(O)N(CH₃)₂, S(O)₂CH₃, S(O)₂NH₂, S(O)₂N(H)CH₃, or S(O)₂N(CH₃)₂; and all other variables are as defined in the second class.

A first sub-class of the third class includes compounds of Formula I and pharmaceutically acceptable salts thereof, wherein all of the variables are as originally defined in the third class; and with the proviso that: (C) AryB is not phenyl that is di-substituted or trisubstituted with O—C₁₋₄ alkyl.

A fourth class of the present invention includes compounds of Formula I and pharmaceutically acceptable salts thereof, wherein R¹ is bromine; and R² is

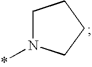

and all other variables are as defined in the second class.

Another embodiment of the present invention is a compound, or a pharmaceutically acceptable salt thereof, selected from the group consisting of the compounds set forth in Examples 1 to 96 below. In an aspect of this embodiment, the compound is selected from the group consisting of the compounds set forth in Examples 1 to 69. In another aspect of this embodiment, the compound is selected from the group consisting of the compounds set forth in Examples 70 to 96.

Another embodiment of the present invention is a compound of Formula I, or a pharmaceutically acceptable salt thereof, as originally defined or as defined in any of the foregoing embodiments, classes, sub-classes, aspects, or features, wherein the compound or its salt is substantially pure. As used herein “substantially pure” means that the compound or its salt is present (e.g., in a product isolated from a chemical reaction or a metabolic process) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The level of purity of the compounds and salts can be determined using standard methods of analysis. A compound or salt of 100% purity can alternatively be described as one which is free of detectable impurities as determined by one or more standard methods of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising an effective amount of Compound I, or a pharmaceutically acceptable salt thereof, and a pharmaceutically acceptable carrier.

(b) A pharmaceutical composition which comprises the product prepared by combining (e.g., mixing) an effective amount of Compound I, or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors other than a compound of Formula I, and HIV integrase inhibitors.

(e) A pharmaceutical combination which is (i) a compound of Formula I, or a pharmaceutically acceptable salt thereof, and (ii) an anti-HIV agent selected from the group consisting of HIV antiviral agents, immunomodulators, and anti-infective agents; wherein the compound of Formula I and the anti-HIV agent are each employed in an amount that renders the combination effective for inhibition of HIV reverse transcriptase, for treatment or prophylaxis of infection by HIV, or for treatment, prophylaxis of, or delay in the onset of AIDS.

(f) The combination of (e), wherein the anti-HIV agent is an antiviral selected from the group consisting of HIV protease inhibitors, HIV reverse transcriptase inhibitors other than a compound of Formula I, and HIV integrase inhibitors.

Additional embodiments of the invention include the pharmaceutical compositions and combinations set forth in (a)-(f) above, wherein the compound of the present invention employed therein is a compound defined in one of the embodiments, classes, or sub-classes described above. In all of these embodiments, the compound can optionally be used, in the form of a pharmaceutically acceptable salt.

Additional embodiments of the present invention include each of the pharmaceutical compositions and combinations set forth in (a)-(f) above and embodiments thereof, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to Compound I or its salt per se; i.e., the purity of the active ingredient in the composition.

The present invention also includes a method for inhibition of HIV reverse transcriptase, for treatment or prophylaxis of HIV infection, or for treatment, prophylaxis of, or delay in the onset of AIDS, which comprises administering to a subject in need thereof an effective amount of a compound of Formula I, or a pharmaceutically acceptable salt thereof, wherein Formula I is as originally set forth and defined above, except that the accompanying proviso A is not applied (i.e., proviso A is absent, but proviso B is still applied). In other words, compounds suitable for use in the method of the present invention include the compounds embraced by Formula I when provisos A and B are applied (i.e., the compounds of the present invention as defined and described above) and the compounds of Formula I that fall within the scope of proviso A but not within the scope of proviso B.

Embodiments of the method of the present invention include those in which the compound of Formula I administered to the subject is as defined in the compound embodiments, classes and sub-classes set forth above, except that any of the provisos A and C to G included therein are not applied. In sub-embodiments of each of these method embodiments, the provisos A to G are applied to the extent they are included in the corresponding compound embodiment, class or sub-class.

The present invention also includes a compound of Formula I, or a pharmaceutically acceptable salt thereof, (i) for use in, (ii) for use as a medicament for, or (iii) for use in the preparation of a medicament for: (a) inhibition of HIV reverse transcriptase, (b) treatment or prophylaxis of infection by HIV, or (c) treatment, prophylaxis of, or delay in the onset of AIDS. In these uses, the compound of Formula I is as originally set forth and defined above, except that the accompanying proviso A is not applied (i.e., proviso A is absent, but proviso B is applied). In these uses, the compounds of the present invention can optionally be employed in combination with one or more anti-HIV agents selected from HIV antiviral agents, anti-infective agents, and immunomodulators. Embodiments of the uses of the present invention include those in which the compound of Formula I is as defined in the compound embodiments, classes and sub-classes set forth above, except that any of provisos A and C to G included therein are not applied. In sub-embodiments of these use embodiments, the provisos A to G are included in the definition of the compound to the extent they are included in the corresponding compound embodiment, class or sub-class.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical (or alternatively an “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest an alkylene selected from the group consisting of —CH₂—, —CH(CH₃)—, and —C(CH₃)₂—.

The term “cycloalkyl” refers to any cyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” appearing in the definition of a functional group (e.g., “C(O)R^{A}”) refers to carbonyl. The term “S(O)₂” or “SO₂” appearing in the definition of a functional group refers to sulfonyl, the term “S(O)” refers to sulfinyl, and the terms “C(O)O” and “CO₂” both refer to carboxyl.

The left-most atom or variable shown in any of the groups in the definitions of R¹ to R⁵ is the atom or variable attached to or nearest to the indole ring. Thus, for example, a compound of the present invention in which R¹ is J-AryA, I in the definition of R¹ is C(O)N(R^{A}), R⁴ is L-CyC, and L is C(O)CH₂, R⁵═H, and R²=phenyl, is as follows:

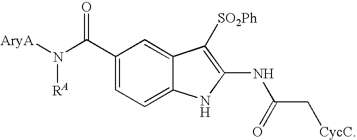

The symbols “*” and “ [symbol] ” at the end of a bond each refer to the point of attachment of a functional group or other chemical moiety to the rest of the molecule of which it is a part.

Unless expressly stated to the contrary in a particular context, any of the various carbocyclic and heterocyclic rings and ring systems defined herein may be attached to the rest of the compound at any ring atom (i.e., any carbon atom or any heteroatom) provided that a stable compound results. Suitable aryls include phenyl, 9- and 10-membered bicyclic, fused carbocyclic ring systems, and 11- to 14-membered tricyclic fused carbocyclic ring systems, wherein in the fused carbocyclic ring systems at least one ring is aromatic. Suitable aryls include, for example, phenyl, naphthyl, tetrahydronaphthyl (tetralinyl), indenyl, anthracenyl, and fluorenyl. Suitable heteroaryls include 5- and 6-membered heteroaromatic rings and 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl (e.g., benzo-1,3-dioxolyl:

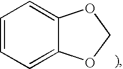

benzopiperidinyl, benzisoxazolyl, benzoxazolyl, chromenyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl (i.e.,

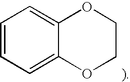

Suitable saturated and mono-unsaturated heterocyclic rings include 4- to 7-membered saturated and mono-unsaturated heterocyclic rings containing at least one carbon atom and from 1 to 4 heteroatoms independently selected from N, O and S, wherein each S is optionally oxidized to S(O) or S(O)₂. Suitable 4- to 7-membered saturated heterocyclics include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Suitable mono-unsaturated heterocyclic rings include those corresponding to the saturated heterocyclic rings listed in the preceding sentence in which a carbon-carbon single bond is replaced with a carbon-carbon double bond (e.g., a carbon-carbon single bond is replaced with a carbon-carbon double bond). Suitable saturated and mono-unsaturated heterobicyclic rings include 6- to 10-membered saturated and mono-unsaturated, bridged or fused heterobicyclic rings containing from 1 to 4 heteroatoms independently selected from N, O and S, where each S is optionally oxidized to S(O) or S(O)₂. Suitable saturated heterobicyclics include those disclosed elsewhere (see, e.g., the definition of HetS in the second class of compounds of the invention), and suitable mono-unsaturated heterobicyclics include those corresponding to the saturated heterobicyclics disclosed elsewhere in which a single bond is replaced with a double bond. It is understood that the specific rings and ring systems suitable for use in the present invention are not limited to those listed in this paragraph. The rings and ring systems listed in this paragraph are merely representative.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, an aryl or heteroaryl, described as optionally substituted with “from 1 to 5 substituents” is intended to include as aspects thereof, an aryl or heteroaryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents.

When any variable (e.g., R^{A}, R^{B}, AryE, or HetE) occurs more than one time in any constituent or in Formula I or in any other formula depicting and describing compounds employed in the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., cycloalkyl, aryl, or heteroaryl) provided such ring substitution is chemically allowed and results in a stable compound. Ring substituents can be attached to the ring atom which is attached to the rest of the molecule; e.g., methyl-substituted 3-oxetanyl refers to:

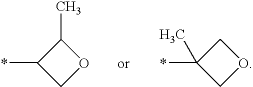

As a result of the selection of substituents and substituent patterns, certain compounds of the present invention can exhibit keto-enol tautomerism. All tautomeric forms of these compounds, whether individually or in mixtures, are within the scope of the present invention. For example, in instances where a hydroxy (—OH) substituent(s) is (are) permitted on a heteroaromatic ring and keto-enol tautomerism is possible, it is understood that the substituent might in fact be present, in whole or in part, in the keto form, as exemplified here for a hydroxypyridinyl substituent:

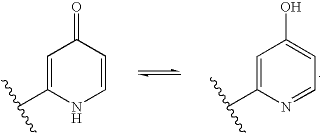

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The method of the present invention involves the use of (i) compounds embraced by Formula I when provisos A and B are applied (i.e., the compounds of the present invention as defined and described above) and (ii) compounds of Formula I that fall within the scope of proviso A but not with the scope of proviso B, in the inhibition of HIV reverse transcriptase (wild type and/or mutant strains thereof), the prophylaxis or treatment of infection by human immunodeficiency virus (HIV) and the prophylaxis, treatment or delay in the onset of consequent pathological conditions such as AIDS. Prophylaxis of AIDS, treating AIDS, delaying the onset of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of a compound of Formula I, or a pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Certain of the compounds employed in the present invention carry an acidic moiety (e.g., —COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV reverse transcriptase, treating or prophylaxis of HIV infection or treating, prophylaxis of, or delaying the onset of AIDS), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to the use of the compounds of Formula I in combination with one or more agents useful in the treatment of HIV infection or AIDS. For example, the compounds of Formula I can be effectively administered, whether at periods of pre-exposure and/or post-exposure, in combination with effective amounts of one or more HIV antiviral agents, immunomodulators, antiinfectives, or vaccines useful for treating HIV infection or AIDS, such as those disclosed in Table 1 of WO 01/38332 or in the Table in WO 02/30930. Suitable HIV antiviral agents for use in combination with the compounds of Formula I include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), non-nucleoside HIV reverse transcriptase inhibitors (e.g., efavirenz or nevirapine), and HIV integrase inhibitors such as those described in WO 02/30930, WO 03/35076, and WO 03/35077. It will be understood that the scope of combinations of compounds of Formula I with HIV antiviral agents, immunomodulators, anti-infectives or vaccines is not limited to the foregoing substances or to the list in the above-referenced Tables in WO 01/38332 and WO 02/30930, but includes in principle any combination with any pharmaceutical composition useful for the treatment of HIV infection or AIDS. The HIV antiviral agents and other agents will typically be employed in these combinations in their conventional dosage ranges and regimens as reported in the art, including, for example, the dosages described in the Physicians' Desk Reference, 58^{th} edition, Thomson PDR, 2004. The dosage ranges for a compound of Formula I in these combinations are the same as those set forth above. It is understood that pharmaceutically acceptable salts of the compounds of the invention and/or the other agents (e.g., indinavir sulfate) can be used as well.

Abbreviations employed herein include the following:
CHAPS=3[(3-cholaraidopropyl)dimethylammonio]-propanesulfonic acid
dGTP=deoxyguanosine triphosphate
DCM=dichloromethane
DIEA=diisopropylethylamine
DMSO=dimethyl sulfoxide
dNTP=deoxynucleoside triphosphate
EDTA=ethylenediaminetetracetic acid
EGTA=ethylene glycol bis(2-aminoethyl ether)-N,N,N′,N′-tetraacetic acid
ES=electrospray
Et=ethyl
i-Pr=isopropyl
LCMS=liquid chromatography mass spectroscopy
MeOH=methanol
MOMCl=methoxymethyl chloride
NMR=nuclear magnetic resonance
Ph=phenyl
PS-DIEA=polystyrene diisopropylethylamine
PS-DMAP=polystyrene 4-N,N-dimethylaminopyridine
PS-DCC=polystyrene dicyclohexylcarbodiimide
Ra-Ni=Raney Nickel
THF=tetrahydrofuran
TFA=trifluoroacteic acid

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 depicts general synthetic routes for preparing many compounds of the present invention. In Part A, suitably substituted 2-chloro-3-sulfonyl-1H-indole 1 (which can be prepared in accordance with procedures set forth in Young et al., Bioorg. Med. Chem. Lett. 1995, 5, 491-496, or routine modifications thereof) can be reacted with hydrazine to obtain indolyl hydrazine 2 which can be reduced (e.g., with Raney Ni) to provide corresponding 2-amino-3-sulfonyl-1H-indole 3. Acylation of 3 with a suitable acylating agent [e.g., treating with an acyl chloride in a suitable solvent (e.g., a halogenated alkane such as dichloromethane) in the presence of a tertiary amine (e.g., triethylamine or DIEA) affords the amide or urea 4. Acylation with a haloalkyl acid halide (e.g., a bromoalkyl acid chloride), followed by nucleophilic displacement of the halogen with a suitable primary or secondary amine furnishes 5. In Part B, after protection of the indole nitrogen with a methoxymethyl group to afford 6, the chloride in 6 can be displaced with various amines which, after removal of the methoxymethyl group, provide 7. In Part C of Scheme 1, the ureas 10 can be prepared from the corresponding ester 8 by saponification to the acid 9 Curtius rearrangement and trapping of the intermediate isocyanate with amines.

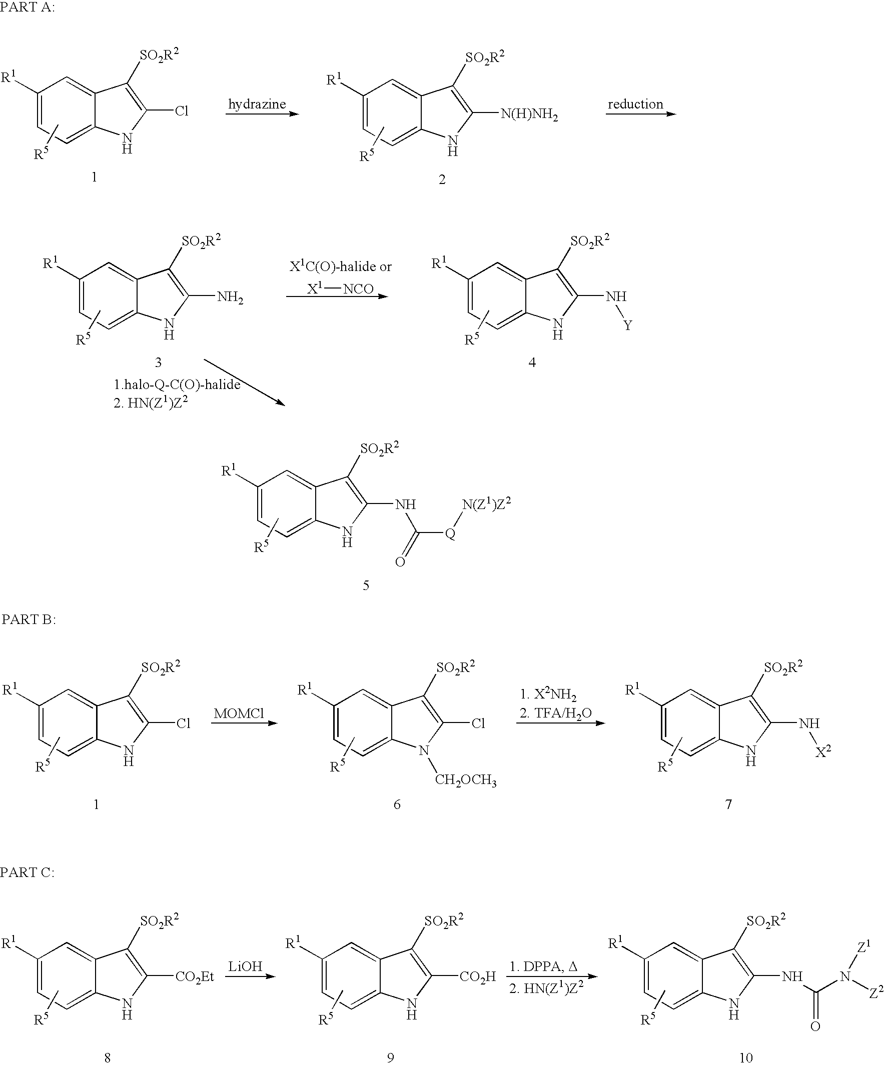

X¹ is (i) alkoxy, (ii) alkyl, cycloalkyl, aryl, or heterocyclyl or (iii) alkyl substituted with cycloalkyl, aryl, or heterocyclyl, wherein any of (i), (ii) or (iii) is optionally substituted.
Y is C(O)—X¹ or C(O)N(H)—X¹.
X² is (i) alkyl, cycloalkyl, aryl, or heterocyclyl or (ii) alkyl substituted with cycloalkyl, aryl, or heterocyclyl, wherein (i) or (ii) is optionally substituted.
Q is linear or branched, optionally substituted, divalent hydrocarbon radical.
Z¹ and Z² are each independently (i) H, (ii) alkyl, cycloalkyl, aryl, or heterocyclyl, or (iii) alkyl substituted with cycloalkyl, aryl, or heterocyclyl, wherein any of (i), (ii), or (ii) is optionally substituted; or Z¹ and Z² together with the N to which they are attached form heterocyclyl which is optionally substituted

In the processes for preparing compounds of the present invention set forth in the foregoing scheme, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention.

### Example 1

### N-[5-chloro-3-(phenylsulfonyl)-1H-indol-2-yl]-2-morpholin-4-ylacetamide

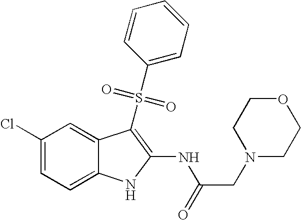

### Step 1: 5-Chloro-2-hydrazino-3-(phenylsulfonyl)-1H-indole

2,5-Dichloro-3-(phenylsulfonyl)-1H-indole (1.0 g, 3.1 mmol) prepared in accordance with Young et al., Bioorg. Med. Chem. Lett. 1995, 5, 491-496) was treated with a solution of 1 M hydrazine in anhydrous THF (40 mL, 40 mmol), and the mixture was stirred at 70° C. for 18 hours. The reaction mixture was cooled to room temperature and concentrated under reduced pressure to afford 5-chloro-2-hydrazino-3-(phenylsulfonyl)-1H-indole, which was used in subsequent steps without further purification. Analytical LCMS: single peak (214 nm), 2.985 min, ES MS (M+H⁺)=322.

### Step 2: 5-Chloro-3-(phenylsulfonyl)-1H-indol-2-amine

A solution of 5-chloro-2-hydrazino-3-(phenylsulfonyl)-1H-indole (1.2 g, 3.7 mmol) in MeOH was treated with Raney Ni (1.5 g wet in MeOH). The reaction mixture was stirred at 70° C. for 1 h before being cooled to room temperature, filtered through a pad of Celite (MeOH wash), and concentrated under reduced pressure to afford 5-chloro-3-(phenylsulfonyl)-1H-indol-2-amine. Analytical LCMS: single peak (214 nm), 2.153 min, ES MS (M+H⁺)=307.

### Step 3: 2-Bromo-N-[5-chloro-3-(phenylsulfonyl)-1H-indol-2-yl]acetamide

A solution of 5-chloro-3-(phenylsulfonyl)-1H-indol-2-amine (1.0 g, 3.3 mmol) in anhydrous CH₂Cl₂ (30 mL) was treated with pyridine (0.54 mL, 6.6 mmol) and bromoacetylbromide (0.43 mL, 5.0 mmol). After stirring for 30 min, the reaction mixture was diluted with H₂O (40 mL) and extracted with CH₂Cl₂ (3×30 mL). The combined organic extracts were dried (Na₂SO₄) and concentrated under reduced pressure solvent to afford 2-bromo-N-[5-chloro-3-(phenylsulfonyl)-1H-indol-2-yl]acetamide which was used in subsequent reactions without further purification. Analytical LCMS: single peak (214 nm), 3.276 min, ES MS (M+H⁺)=427.

### Step 4: N-[5-chloro-3-(phenylsulfonyl)-1H-indol-2-yl]-2-morpholin-4-ylacetamide

A solution of 2-bromo-N-[5-chloro-3-(phenylsulfonyl)-1H-indol-2-yl]acetamide (10 mg, 0.023 mmol) in anhydrous CH₂Cl₂ was treated with morpholine (0.1 mL, 1.14 mmol) and stirred at room temperature. After 10 min, the reaction was evaporated under a stream of N₂ and purified by reverse phase chromatography to afford N-[5-chloro-3-(phenylsulfonyl)-1H-indol-2-yl]-2-morpholin-4-ylacetamide. Analytical LCMS: single peak (214 nm), 2.879; 1H NMR (CDCl₃, 300 MHz) δ 10.95 (s, 1H), 10.92 (s, 1H), 7.94 (d, J=7.2 Hz, 2H), 7.65 (d, J=1.8 Hz, 1H), 7.57-7.48 (m, 3H), 7.24 (d, J=9 Hz, 1H), 7.17 (dd, J=1.8, 8.7 Hz, 1H), 4.01 (m, 4H), 3.90 (s, 2H), 3.17 (m, 4H); HRMS m/z 434.0920 (C₂₀H₂₀ClN₃O₄S+H⁺ requires 434.0936).

### Example 2

### N-[5-chloro-3-(phenylsulfonyl)-1H-indol-2-yl]-N-(2-fluorophenyl)urea

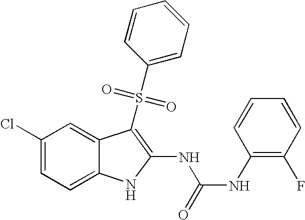

### Step 1: N-[5-chloro-3-(phenylsulfonyl)-1H-indol-2-yl]-N-(2-fluorophenyl)urea

A solution of 5-chloro-3-(phenylsulfonyl)-1H-indol-2-amine (20 mg, 0.065 mmol) in anhydrous CH₂Cl₂ (2 mL) was treated with i-Pr₂NEt (0.2 mL, 1.2 mmol) and 2-fluorophenylisocyanate (0.05 mL, 0.36 mmol). After stirring for 30 min, the reaction mixture was evaporated under a stream of N₂ and purified by reverse phase chromatography to afford N-[5-chloro-3-(phenylsulfonyl)-1H-indol-2-yl]-N-(2-fluorophenyl)urea. Analytical LCMS: single peak (214 nm), 3.594 min; ¹H NMR (CDCl₃, 300 MHz) δ 9.16 (br s, 2H); 8.14 (dd, J=8.1, 8.1 Hz, 2H); 7.62-7.48 (m, 2H); 7.42-7.32 (m, 3H); 7.16-7.04 (m, 6H); HRMS m/z 444.0588 (C₂₁H₁₅ClFN₃O₃S+H⁺ requires 443.0580).

### Example 3

### N-[5-Chloro-3-(phenylsulfonyl)-1H-indol-2-yl]cyclopropanecarboxamide

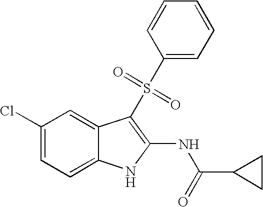

A mixture of 5-chloro-3-(phenylsulfonyl)-1H-indol-2-amine (31 mg, 0.1 mmol), cyclopropanecarbonyl chloride (15 mg, 0.15 mmol), and pyridine (100 μL) in DCM (1 mL) was heated at 45° C. for 2 hours. After this time, the solution was concentrated under a nitrogen blower. The concentrated residue was purified by LCMS to give the desired product as a slightly yellow solid. Analytical LCMS: single peak (214 nm), 2.758 min, ES MS (M+1)=375.1; ¹H NMR (500 MHz, d₆-DMSO) δ 12.51 (br s, 1H), 10.33 (s, 1H), 8.06-8.03 (m, 2H), 7.62-7.58 (m, 3H), 7.56 (d, J=2.0 Hz, 1H), 7.53 (d, J=8.5 Hz, 1H), 7.17 (dd, J=8.5, 2.0 Hz, 1H), 2.31-2.24 (m, 1H), 1.05-0.93 (m, 4H); HRMS, calc'd for C₈H₁₆ClN₂O₃S (M+H), 375.0565; found 375.0575.

### Examples 4-69

In the following table, the amide compounds were prepared using procedures similar to those employed in Examples 1 and 3, and the urea compounds were prepared using procedures similar to those employed in Example 2.

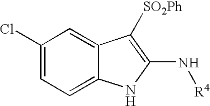

| | | | ES MS |
|---|---|---|---|
| Ex. | Name | R⁴ | (M + 1) |
| 4 | 2-(4-chlorophenoxy)-N-[5-chloro-3- (phenylsulfonyl)-1H-indol-2- yl]acetamide | 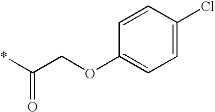 | 476.4 |
| 5 | 2-{[5-chloro-3-(phenylsulfonyl)-1H- | C(O)CH₂OC(O)CH₃ | 407.8 |
| | indol-2-yl]amino}-2-oxoethyl acetate | | |
| 6 | 2-(benzyloxy)-N-[5-chloro-3- (phenylsulfonyl)-1H-indol-2- yl]acetamide | 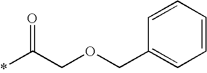 | 455.9 |
| 7 | N-[5-chloro-3-(phenylsulfonyl)-1H- | C(O)CH₂CH₃ | 363.8 |
| | indol-2-yl]propanamide | | |
| 8 | N-[5-chloro-3-(phenylsulfonyl)-1H- indol-2-yl]-3-phenoxypropanamide | 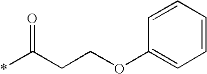 | 455.9 |
| 9 | N-[5-chloro-3-(phenylsulfonyl)-1H- indol-2-yl]cyclobutanecarboxamide | 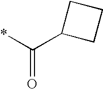 | 389.9 |
| 10 | N-[5-chloro-3-(phenylsulfonyl)-1H- indol-2-yl]-2,3-dihydro-1-benzofuran- 2-carboxamide | 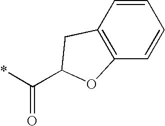 | 453.9 |
| 11 | N-[5-chloro-3-(phenylsulfonyl)-1H- indol-2-yl]-2,3-dihydro-1,4- benzodioxine-2-carboxamide | 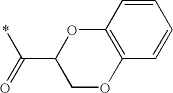 | 469.9 |
| 12 | N¹-[5-chloro-3-(phenylsulfonyl)-1H- indol-2-yl]-N²- cyclopropylglycinamide | 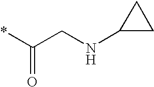 | 404.9 |
| 13 | N¹-[5-chloro-3-(phenylsulfonyl)-1H- indol-2-yl]-N²-(pyridin-4- ylmethyl)glycinamide | 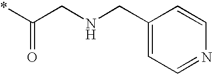 | 455.9 |
| 14 | N-[5-chloro-3-(phenylsulfonyl)-1H- indol-2-yl]nicotinamide | 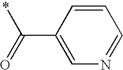 | 412.9 |
| 15 | N-[5-chloro-3-(phenylsulfonyl)-1H- | C(O)CH(CH₃)₂ | 377.9 |
| | indol-2-yl]-2-methylpropanamide | | |
| 16 | N-[5-chloro-3-(phenylsulfonyl)-1H- indol-2-yl]-2-(4- fluorophenyl)acetamide | 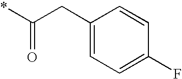 | 443.9 |
| 17 | N-[5-chloro-3-(phenylsulfonyl)-1H- indol-2-yl]-2-(3,3-difluoropiperidin- 1-yl)acetamide | 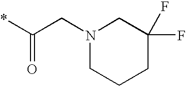 | 468.9 |
| 18 | N-[5-chloro-3-(phenylsulfonyl)-1H- indol-2-yl]-2,4-difluorobenzamide | 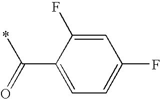 | 447.9 |
| 19 | N-[5-chloro-3-(phenylsulfonyl)-1H- indol-2-yl]-2-fluorobenzamide | 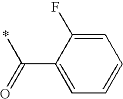 | 429.9 |
| 20 | N-[5-chloro-3-(phenylsulfonyl)-1H- indol-2-yl]isonicotinamide | 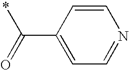 | 412.9 |
| 21 | N¹-[5-chloro-3-(phenylsulfonyl)-1H- | C(O)CH₂N(H)CH₂CH₃ | 392.9 |
| | indol-2-yl]-N²-ethylglycinamide | | |
| 22 | N-[5-chloro-3-(phenylsulfonyl)-1H- indol-2-yl]-4-cyanobenzamide | 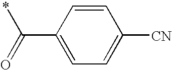 | 436.9 |
| 23 | N²-benzyl-N¹-[5-chloro-3- (phenylsulfonyl)-1H-indol-2- yl]glycinamide | 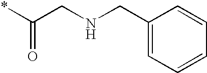 | 455.0 |
| 24 | N-[5-chloro-3-(phenylsulfonyl)-1H- indol-2-yl]-3-methyl-2-furamide | 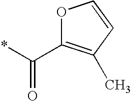 | 415.9 |
| 25 | N-[5-chloro-3-(phenylsulfonyl)-1H- indol-2-yl]-3-fluorobenzamide | 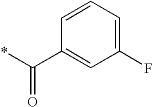 | 429.9 |
| 26 | N-[5-chloro-3-(phenylsulfonyl)-1H- | C(O)C(CH₃)CH₂CH₃ | 391.9 |
| | indol-2-yl]-2-methylbutanamide | | |
| 27 | ethyl N-({[5-chloro-3- | C(O)N(H)CH₂C(O)OCH₂CH₃ | 436.9 |
| | (phenylsulfonyl)-1H-indol-2- | | |
| | yl]amino}carbonyl)glycinate | | |
| 28 | N-[5-chloro-3-(phenylsulfonyl)-1H- indol-2-yl]benzamide | 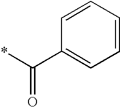 | 411.9 |
| 29 | N-[5-chloro-3-(phenylsulfonyl)-1H- indol-2-yl]-N′-(3-fluorophenyl)urea | 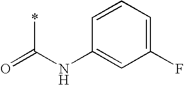 | 444.9 |
| 30 | N¹-[5-chloro-3-(phenylsulfonyl)-1H- indol-2-yl]-N′~2~-(2- furylmethyl)glycinamide | 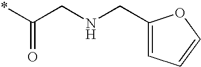 | 444.9 |
| 31 | N-[5-chloro-3-(phenylsulfonyl)-1H- indol-2-yl]-N′-(4-fluorophenyl)urea | 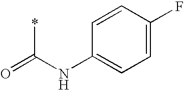 | 444.9 |
| 32 | N¹-[5-chloro-3-(phenylsulfonyl)-1H- indol-2-yl]-N²-(pyridin-3- ylmethyl)glycinamide | 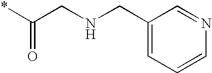 | 455.9 |
| 33 | N¹-[5-chloro-3-(phenylsulfonyl)-1H- indol-2-yl]-N²-(isoxazol-3- ylmethyl)glycinamide | 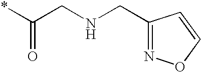 | 445.9 |
| 34 | N-[5-chloro-3-(phenylsulfonyl)-1H- | C(O)CH₂OCH₃ | 379.8 |
| | indol-2-yl]-2-methoxyacetamide | | |
| 35 | N-[5-chloro-3-(phenylsulfonyl)-1H- indol-2-yl]-N′-phenylurea | 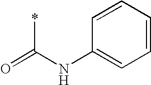 | 426.9 |
| 36 | N¹-[5-chloro-3-(phenylsulfonyl)-1H- indol-2-yl]-N²-(1-pyridin-4- ylethyl)glycinamide | 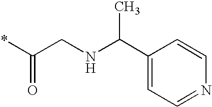 | 470.0 |
| 37 | N-[5-chloro-3-(phenylsulfonyl)-1H- indol-2-yl]-2-(4-pyridin-4- ylpiperidin-1-yl)acetamide | 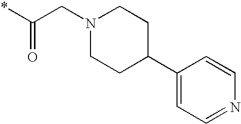 | 510.0 |
| 38 | N¹-[5-chloro-3-(phenylsulfonyl)-1H- indol-2-yl]-N²-(1,3-thiazol-4- ylmethyl)glycinamide | 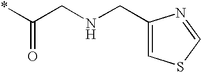 | 462.0 |
| 39 | (2R)-N-[5-chloro-3-(phenylsulfonyl)- 1H-indol-2-yl]-3,3,3-trifluoro-2- methoxy-2-phenylpropanamide | 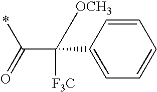 | 523.9 |
| 40 | N-[5-chloro-3-(phenylsulfonyl)-1H- indol-2-yl]-2-[4-(pyridin-2- ylmethyl)piperazin-1-yl]acetamide | 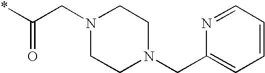 | 525.0 |
| 41 | N-[5-chloro-3-(phenylsulfonyl)-1H- indol-2-yl]-N′-[2- (trifluoromethyl)phenyl]urea | 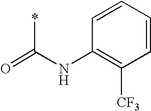 | 494.9 |
| 42 | N¹-[5-chloro-3-(phenylsulfonyl)-1H- indol-2-yl]-N²-[(1-methyl-1H- imidazol-2-yl)methyl]glycinamide | 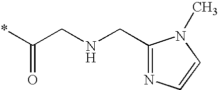 | 458.9 |
| 43 | N-[5-chloro-3-(phenylsulfonyl)-1H- indol-2-yl]-N′-(3-methylbenzyl)urea | 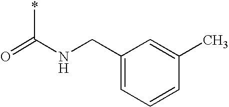 | 455.0 |
| 44 | N-[5-chloro-3-(phenylsulfonyl)-1H- indol-2-yl]-N′-cyclopentylurea | 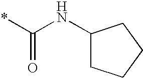 | 419.0 |
| 45 | N¹-[5-chloro-3-(phenylsulfonyl)-1H- indol-2-yl]-N²-[(3-methyloxetan-3- yl)methyl]glycinamide | 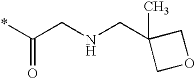 | 448.9 |
| 46 | N-(sec-butyl)-N′-[5-chloro-3- | C(O)N(H)CH(CH₃)CH₂CH₃ | 406.9 |
| | (phenylsulfonyl)-1H-indol-2-yl]urea | | |
| 47 | N-[5-chloro-3-(phenylsulfonyl)-1H- indol-2-yl]cyclopentanecarboxamide | 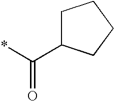 | 403.9 |
| 48 | N-butyl-N′-[5-chloro-3- | C(O)N(H)CH₂CH₂CH₂CH₃ | 406.9 |
| | (phenylsulfonyl)-1H-indol-2-yl]urea | | |
| 49 | N-[5-chloro-3-(phenylsulfonyl)-1H- indol-2-yl]-N′-(2-phenylethyl)urea | 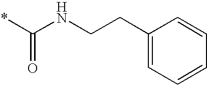 | 455.0 |
| 50 | N-[5-chloro-3-(phenylsulfonyl)-1H- indol-2-yl]-N′-(3-fluorobenzyl)urea | 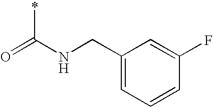 | 458.9 |
| 51 | N¹-[5-chloro-3-(phenylsulfonyl)-1H- indol-2-yl]-N²-(3- methoxybenzyl)glycinamide | 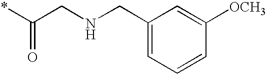 | 458.0 |
| 52 | N-[5-chloro-3-(phenylsulfonyl)-1H- indol-2-yl]-N′-(4-fluorobenzyl)urea | 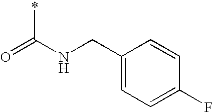 | 458.9 |
| 53 | N-[5-chloro-3-(phenylsulfonyl)-1H- indol-2-yl]-2-[4- (methylsulfonyl)piperazin-1- yl]acetamide | 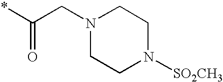 | 512.0 |
| 54 | N¹-[5-chloro-3-(phenylsulfonyl)-1H- indol-2-yl]-N²-(1-pyridin-3- ylethyl)glycinamide | 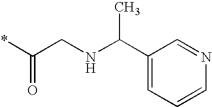 | 470.0 |
| 55 | N¹-[5-chloro-3-(phenylsulfonyl)-1H- | C(O)CH₂NH(CH₂)₅OH | 451.0 |
| | indol-2-yl]-N²-(5- | | |
| | hydroxypentyl)glycinamide | | |
| 56 | N-[5-chloro-3-(phenylsulfonyl)-1H- indol-2-yl]-2-(3-pyridin-2- ylpyrrolidin-1-yl)acetamide | 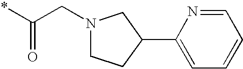 | 496.0 |
| 57 | N-[5-chloro-3-(phenylsulfonyl)-1H- indol-2-yl]-N′-cyclohexylurea | 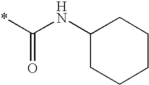 | 432.9 |
| 58 | N-[5-chloro-3-(phenylsulfonyl)-1H- indol-2-yl]-N′-(2- phenylcyclopropyl)urea | 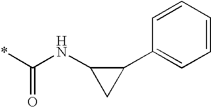 | 467.0 |
| 59 | 2-{[5-chloro-3-(phenylsulfonyl)-1H- indol-2-yl]amino}-2-oxo-N-(1- phenylethyl)ethanamine | 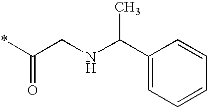 | 469.0 |
| 60 | N-[5-chloro-3-(phenylsulfonyl)-1H- indol-2-yl]cyclohexanecarboxamide | 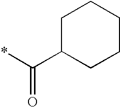 | 417.9 |
| 61 | N-[5-chloro-3-(phenylsulfonyl)-1H- indol-2-yl]-2-(4-methylpiperazin-1- yl)acetamide | 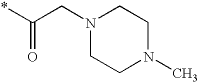 | 448.0 |
| 62 | N-[5-chloro-3-(phenylsulfonyl)-1H- | C(O)N(H)CH(CH₃)₂ | 392.9 |
| | indol-2-yl]-N′-isopropylurea | | |
| 63 | N-[5-chloro-3-(phenylsulfonyl)-1H- indol-2-yl]-2-furamide | 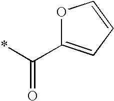 | 401.8 |
| 64 | N¹-[5-chloro-3-(phenylsulfonyl)-1H- indol-2-yl]-N²-ethyl-N~2~-(pyridin- 4-ylmethyl)glycinamide | 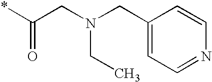 | 484.0 |
| 65 | N-[5-chloro-3-(phenylsulfonyl)-1H- indol-2-yl]-2-phenoxyacetamide | 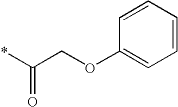 | 441.9 |
| 66 | N-[5-chloro-3-(phenylsulfonyl)-1H- indol-2-yl]-N′-(3,5- difluorophenyl)urea | 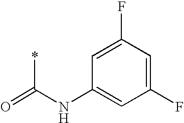 | 462.9 |
| 67 | N-[5-chloro-3-(phenylsulfonyl)-1H- indol-2-yl]-2-[4-(5-methoxypyridin-2- yl)piperazin-1-yl]acetamide | 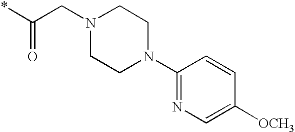 | 541.0 |
| 68 | N-[5-chloro-3-(phenylsulfonyl)-1H- indol-2-yl]-N′-[3- (trifluoromethyl)phenyl]urea | 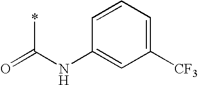 | 494.9 |
| 69 | N′-(2-{[5-chloro-3-(phenylsulfonyl)- | C(O)CH₂N(H)CH₂CH₂N(CH₂CH₃)₂ | 464.0 |
| | 1H-indol-2-yl]amino}-2-oxoethyl)- | | |
| | N,N-diethylethane-1,2-diamine | | |

### Example 70

### N-[5-bromo-3-(pyrrolidin-1-ylsulfonyl)-1H-indol-2-yl]-N-(3-fluorobenzyl)urea

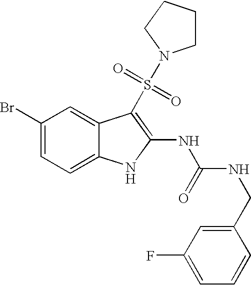

### Step 1: Ethyl 5-bromo-3-(pyrrolidin-1-ylsulfonyl)-1H-indole-2-carboxylate

Pyrrolidine (1820 μL, 21.0 mmol) was added to a solution of ethyl 5-bromo-3-(chlorosulfonyl)-1-(phenylsulfonyl)-1H-indole-2-carboxylate (3.57 g, 7.0 mmol) and pyridine (1400 μL, 14 mmol) in DCM (50 mL) at 0° C. with stirring. The resultant mixture solution was stirred from 0° C. to room temperature for 16 hours. After this time, the solution was diluted with DCM (50 mL) and washed with 1N HCl (3×50 mL), brine (50 mL), dried over Na₂SO₄, filtered, and concentrated. The concentrated residue was purified by LCMS to give the desired product ethyl 5-bromo-3-(pyrrolidin-1-ylsulfonyl)-1H-indole-2-carboxylate as a slightly yellow solid. Analytical LCMS: single peak (214 nm), 3.273 min, ES MS (M+1)=401.

### Step 2: 5-Bromo-3-(pyrrolidin-1-ylsulfonyl)-1H-indole-2-carboxylic acid

A mixture of ethyl 5-bromo-3-(pyrrolidin-1-ylsulfonyl)-1H-indole-2-carboxylate (1.61 g, 4.0 mmol) and LiOH (500 mg,) in THF/MeOH/H2O (2:2:1, 50 mL) was heated at 70° C. for 4 hours. After this time, the solution was concentrated to a small volume and then treated with 1N HCl to adjust the solution pH to about 2. The slightly yellow precipitate was collected by filtration and washed with water (3×10 mL). After drying, analytical LCMS confirmed that this yellow solid was the desired pure product 5-bromo-3-(pyrrolidin-1-ylsulfonyl)-1H-indole-2-carboxylic acid. Analytical LCMS: single peak (214 nm), 2.937 min, ES MS (M+1)=373.

### Step 3: N-[5-bromo-3-(pyrrolidin-1-ylsulfonyl)-1H-indol-2-yl]-N-(3-fluorobenzyl)urea

A mixture of 5-bromo-3-(pyrrolidin-1-ylsulfonyl)-1H-indole-2-carboxylic acid (27 mg, 0.07 mmol), diphenylphosphorylazide (0.047 mL, 0.22 mmol), and triethylamine (0.03 mL, 0.22 mmol) in benzene (2.7 mL) was irradiated at 60° C. for 30 min in an Emrys Optimizer microwave reactor. After cooling the reaction mixture to room temperature, 3-fluorobenzylamine (0.075 mL, 0.65 mmol) was added and the mixture warmed at 60° C. in a heat block. After 18 hours, the reaction was cooled to room temperature, evaporated under reduced pressure, and purified by reverse phase HPLC to afford N-[5-bromo-3-(pyrrolidin-1-ylsulfonyl)-1H-indol-2-yl]-N-(3-fluorobenzyl)urea. Analytical LCMS: single peak (214 nm), 3.608 min; ¹H NMR (CDCl₃, 300 MHz) δ 12.98 (s, 1H), 9.34 (t, J=5.4 Hz, 1H), 8.09 (m, 1H), 7.52 (d, J=9 Hz, 1H), 7.47 (dd, J=1.8, 9 Hz, 1H), 7.40 (ddd, J=6.0, 7.8, 14.1 Hz, 1H), 7.26 (m, 2H), 7.11 (dt, J=2.7, 9.0 Hz, 1H), 4.57 (d, J=5.7 Hz, 2H), 3.14 (m, 4H), 1.63 (m, 4H); HRMS m/z 495.0506 (C₂₀H₂₀BrFN₄O₃S+H⁺ requires 495.0497).

### Examples 71-96

In the following table, the amide compounds were prepared using procedures similar to those employed in Example 4.

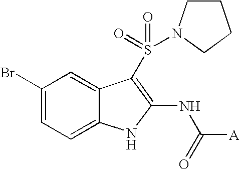

| | | | ES MS |
|---|---|---|---|
| Ex. | Name | A | (M + 1) |
| 71 | N-[5-bromo-3-(pyrrolidin-1-ylsulfonyl)- 1H-indol-2-yl]-N′-(3-chlorobenzyl)urea | 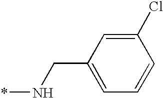 | 512.8 |
| 72 | N-benzyl-N′-[5-bromo-3-(pyrrolidin-1- ylsulfonyl)-1H-indol-2-yl]urea | 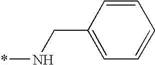 | 478.4 |
| 73 | N-[5-bromo-3-(pyrrolidin-1-ylsulfonyl)- 1H-indo1-2-yl]-N′-phenylurea | 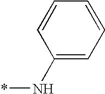 | 464.4 |
| 74 | N-[5-bromo-3-(pyrrolidin-1-ylsulfonyl)- | N(H)CH(CH₃)₂ | 430.3 |
| | 1H-indol-2-yl]-N′-isopropylurea | | |
| 75 | N-[5-bromo-3-(pyrrolidin-1-ylsulfonyl)- 1H-indol-2-yl]-N′-pyridin-2-ylurea | 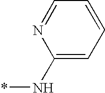 | 465.3 |
| 76 | N-[5-bromo-3-(pyrrolidin-1-ylsulfonyl)- 1H-indol-2-yl]-N-cyclopropylurea | 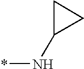 | 428.3 |
| 77 | N-[5-bromo-3-(pyrrolidin-1-ylsulfonyl)- 1H-indol-2-yl]-N′-(2,6- difluorophenyl)urea | 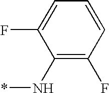 | 500.3 |
| 78 | N-[5-bromo-3-(pyrrolidin-1-ylsulfonyl)- 1H-indol-2-yl]-N′-cyclopentylurea | 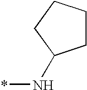 | 456.4 |
| 79 | N-[5-bromo-3-(pyrrolidin-1-ylsulfonyl)- 1H-indol-2-yl]-N′-(2-hydroxybenzyl)urea | 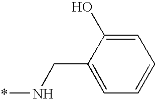 | 494.4 |
| 80 | N-[5-bromo-3-(pyrrolidin-1-ylsulfonyl)- 1H-indol-2-yl]-N′-(pyridin-2- ylmethyl)urea | 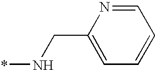 | 479.4 |
| 81 | N-[5-bromo-3-(pyrrolidin-1-ylsulfonyl)- 1H-indol-2-yl]-N′-(pyridin-3- ylmethyl)urea | 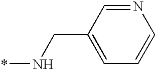 | 479.4 |
| 82 | N-[5-bromo-3-(pyrrolidin-1-ylsulfonyl)- | N(H)CH₂CH₃ | 416.3 |
| | 1H-indol-2-yl]-N-ethylurea | | |
| 83 | N-[5-bromo-3-(pyrrolidin-1-ylsulfonyl)- 1H-indol-2-yl]-N′-(1,3-thiazol-5- ylmethyl)urea | 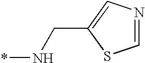 | 485.4 |
| 84 | N-[5-bromo-3-(pyrrolidin-1-ylsulfonyl)- 1H-indol-2-yl]pyrrolidine-1-carboxamide | 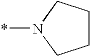 | 442.4 |
| 85 | N-[5-bromo-3-(pyrrolidin-1-ylsulfonyl)- 1H-indol-2-yl]-N′-(2-phenylethyl)urea | 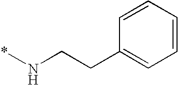 | 492.4 |
| 86 | N-[5-bromo-3-(pyrrolidin-1-ylsulfonyl)- 1H-indol-2-yl]-N′-(pyridin-4- ylmethyl)urea | 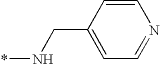 | 479.4 |
| 87 | N¹-[5-bromo-3-(pyrrolidin-1-ylsulfonyl)- 1H-indol-2-yl]piperidine-1,3- dicarboxamide | 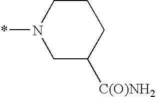 | 499.4 |
| 88 | N-[5-bromo-3-(pyrrolidin-1-ylsulfonyl)- 1H-indol-2-yl]-N′-(2-pyridin-2- ylethyl)urea | 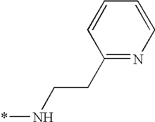 | 493.4 |
| 89 | N-[5-bromo-3-(pyrrolidin-1-ylsulfonyl)- | NH₂ | 388.3 |
| | 1H-indol-2-yl]urea | | |
| 90 | N-[5-bromo-3-(pyrrolidin-1-ylsulfonyl)- | N(H)CH₂CH₂CH₂-Ph | 506.4 |
| | 1H-indol-2-yl]-N′-(3-phenylpropyl)urea | | |
| 91 | N-[5-bromo-3-(pyrrolidin-1-ylsulfonyl)- 1H-indol-2-yl]-4-methylpiperazine-1- carboxamide | 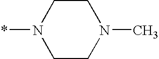 | 471.4 |
| 92 | N-[5-bromo-3-(pyrrolidin-1-ylsulfonyl)- | N(H)CH₂CH₂C(CH₃)₃ | 472.4 |
| | 1H-indol-2-yl]-N′-(3,3- | | |
| | dimethylbutyl)urea | | |
| 93 | N-(2-anilinoethyl)-N′-[5-bromo-3- (pyrrolidin-1-ylsulfonyl)-1H-indol-2- yl]urea | 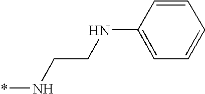 | 507.4 |
| 94 | N-[5-bromo-3-(pyrrolidin-1-ylsulfonyl)- | N(H)CH₂CH₂CH₂N(CH₃)₂ | 473.4 |
| | 1H-indol-2-yl]-N′-[3- | | |
| | (dimethylamino)propyl]urea | | |
| 95 | N-[5-bromo-3-(pyrrolidin-1-ylsulfonyl)- 1H-indol-2-yl]-N′-(2-chloro-6- fluorobenzyl)urea | 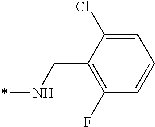 | 530.8 |
| 96 | N-[5-bromo-3-(pyrrolidin-1-ylsulfonyl)- 1H-indol-2-yl]azetidine-1-carboxamide | 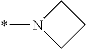 | 428.3 |

### Example 97

### Encapsulated Oral Compositions

A capsule formulation suitable for use in the present invention can be prepared by filling standard two-piece gelatin capsules each with 100 mg of the title compound of Example 1, 150 mg of lactose, 50 mg of cellulose, and 3 mg of stearic acid. Encapsulated oral compositions containing any one of the title compounds of Examples 2 to 96 can be similarly prepared.

### Example 98

### Assay for Inhibition of HIV Reverse Transcriptase

An assay to determine the in vitro inhibition of FEN reverse transcriptase by compounds of the present invention was conducted as follows: HIV-1 RT enzyme (1 nM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microliter Optiplates (Packard). 100 μL reaction mixtures were initiated with a combination of primer-template substrate (10 nM final concentration) and dNTPs (0.6 μM dNTPs, 0.75 μM [³H]-dGTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μl, streptavidin scintillation proximity assay beads (10 mg/mL, from Amersham Biosciences) in 0.5 M EDTA, pH 8. Microtiter plates were incubated an additional 10 minutes at 37° C. prior to quantification via Topcount (Packard). Representative compounds of the present invention exhibit inhibition of the reverse transcriptase enzyme in this assay. For example, the title compounds set forth above in Examples 1 to 96 were tested in the assay and all were found to have IC₅₀ values of less than 1 micromolar.

Analogous assays were conducted substituting mutant HIV strains to determine the in vivo inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Certain compounds of the present invention exhibit inhibition of the reverse transcriptase enzyme in these assays. For example, in the Y181C mutant assay the compounds set forth above in Examples 5, 10, 11, 21, 26, 38, 39 and 72 were found to have IC₅₀ values of less than 1 micromolar, and the compounds of Examples 3, 15, 71, 75-77, 80, 81, 83 and 94 were found to have IC₅₀ values of greater than 1 micromolar and less than 20 micromolar. The compounds of Examples 8, 31, 37, 78, 79, 82 and 84-93 were tested in the Y181C assay up to 20 micromolar, but specific IC₅₀ values were not obtained; i.e., the IC₅₀ values were greater than 20 micromolar. The compounds set forth in the other Examples were not tested in the Y181C assay. In the K103N mutant assay, the compounds of Examples 2, 3, 5, 10, 11, 21, 38, 39, 70-87, 89, 90 and 94 were found to have IC₅₀ values of less than 1 micromolar, and the compounds of Examples 15, 26, 88, 91 and 93 were found to have IC₅₀ values of greater than 1 micromolar and less than 20 micromolar. The compounds of Examples 8, 31, 37 and 92 were tested in the K103N assay up to 20 micromolar, but specific IC₅₀ values were not obtained; i.e., the IC₅₀ values were greater than 20 micromolar. The compounds set forth in the other Examples were not tested in the K103N assay.

### Example 99

### Assay for Inhibition of HIV Replication

An assay for the inhibition of acute HIV infection of T-lymphoid cells (alternatively referred to herein as the “spread assay”) was conducted in accordance with Vacca, J. P. et al., Proc. Natl. Acad. Sci. USA 1994, 91: 4096. Representative compounds of the present invention exhibit inhibition of HIV replication in this assay. For example, the compounds set forth in Examples 2, 3, 5-12, 15-21, 24-31, 34, 35, 39, 41-48, 50-53, 56-62, 65, 66, 68, 71-73, 75, 77, 86, 89 and 95 were found to have IC₉₅ values of less than 1 micromolar, and the compounds of Examples 49, 90 and 96 were found to have IC₉₅ values of greater than 1 micromolar and less than 10 micromolar. The compounds set forth in the other Examples were not tested in the spread assay.

### Example 100

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 99) were examined for cytotoxicity. For those compounds for which an IC₉₅ value was determined in the spread assay, no cytotoxicity was exhibited at the IC₉₅ concentration; i.e., their toxicity value is greater than their IC₉₅ value. In particular, the compounds set forth in Examples 2, 3, 5-12, 15-21, 24-31, 34, 35, 39, 41-53, 56-62, 65, 66, 68, 71-73, 75, 77, 86, 89, 90, 95 and 96 exhibited no cytotoxicity at their IC₉₅ concentrations.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A compound of Formula I, or a pharmaceutically acceptable salt thereof: 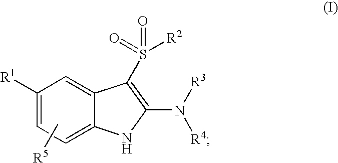
wherein:
R¹ is:
(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)N(R^{A})R^{B},
(17) N(R^{A})C(O)N(R^{A})R^{B},
(18) C₁₋₆ alkyl,
(19) C₁₋₆ haloalkyl,
(20) C₂₋₆ alkenyl,
(21) C₂₋₆ alkynyl,
(22) OH,
(23) O—C₁₋₆ alkyl,
(24) O—C₁₋₆ haloalkyl,
(25) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(26) CycA,
(27) AryA,
(28) HetA,
(29) HetR,
(30) C₁₋₆ alkyl substituted with CycA, AryA, HetA, or HetR,
(31) J-CycA,
(32) J-AryA,
(33) J-HetA, or
(34) J-HetR;
J is:
(1) O,
(2) S,
(3) S(O),
(4) S(O)₂,
(5) O—C₁₋₆ alkylene,
(6) S—C₁₋₆ alkylene,
(7) S(O)—C₁₋₆ alkylene,
(8) S(O)₂—C₁₋₆ alkylene,
(9) N(R^{A}),
(10) N(R^{A})—C₁₋₆ alkylene,
(11) C(O),
(12) C(O)—C₁₋₆ alkylene-O,
(13) C(O)N(R^{A}),
(14) C(O)N(R^{A})—C₁₋₆ alkylene,
(15) C(O)N(R^{A})—C₁₋₆ alkylene-C(O)O, or
(16) C(O)N(R^{A})S(O)₂;
CycA is C₃₋₈ cycloalkyl which is optionally substituted with a total of from 1 to 6 substituents, wherein:
(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
AryA is aryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B}, or
(25) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
HetA is heteroaryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R¹³,
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
HetR is (i) a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, or (ii) a 6- to 10-membered saturated or mono-unsaturated, bridged or fused heterobicyclic ring containing from 1 to 4 heteroatoms independently selected from N, O and S, where each S is optionally oxidized to S(O) or S(O)₂; and wherein the saturated or mono-unsaturated heterocyclic or heterobicyclic ring is optionally substituted with a total of from 1 to 4 substituents, wherein:
(i) from zero to 4 substituents are each independently halogen, CN, C₁₋₆ alkyl, OH, oxo, C(O)R^{A}, C(O)OR^{A}, C(O)N(R^{A})R^{B}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, AryE, HetE, HetF, or C₁₋₆ alkyl substituted with CycE, AryE, HetE, or HetF;
R² is:
(1) C₁₋₆ alkyl,
(2) C₁₋₆ haloalkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(3) CycB,
(4) AryB,
(5) HetB,
(6) HetS,
(7) C₁₋₆ alkyl substituted with CycB, AryB, HetB, or HetS,
(8) N(R^{A})—C₁₋₆ allyl,
(9) N(R^{A})—C₁₋₆ alkyl, wherein the alkyl is substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(10) N(R^{A})-CycB,
(11) N(R^{A})-AryB,
(12) N(R^{A})-HetB, or
(13) N(R^{A})—C₁₋₆ alkyl, wherein the alkyl is substituted with CycB, AryB, HetB, or HetS;
CycB independently has the same definition as CycA;
AryB independently has the same definition as AryA;
HetB independently has the same definition as HetA;
HetS independently has the same definition as HetR;
R³ is H or C₁₋₆ alkyl;
R⁴ is:
(1) H,
(2) N(H)R^{A},
(3) C₁₋₆ alkyl,
(4) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(5) C₁₋₆ haloalkyl,
(6) C(O)—C₁₋₆ alkyl,
(7) C(O)—C₁₋₆ alkylene-O—C₁₋₄ alkyl,
(8) C(O)—C₁₋₆ alkylene-O(C═O)—C₁₋₆ alkyl,
(9) C(O)—C₁₋₆ alkylene-C(O)O—C₁₋₆ alkyl,
(10) C(O)—C₁₋₆ alkylene-N(R^{A})R^{B},
(11) C(O)—C₁₋₆ alkylene-N(R^{A})—C₂₋₆ alkylene-OH, with the proviso that the OH is not attached to the carbon in C₂₋₆ alkylene that is directly attached to the rest of the molecule,
(12) C(O)—C₁₋₆ alkylene-N(R^{A})—C₁₋₆ alkylene-N(R^{A})R^{B},
(13) C(O)—O—C₁₋₆ alkyl,
(14) C(O)N(R^{A})R^{B},
(15) C(O)N(R^{A})—C₁₋₆ alkylene-N(R^{A})R^{B},
(16) C(O)N(R^{A})—C₁₋₆ allylene-C(O)—O—C₁₋₆ alkyl,
(17) SO₂R^{A},
(18) SO₂N(R^{A})R^{B},
(19) C₂₋₆ alkenyl,
(20) C₂₋₆ alkynyl,
(21) CycC,
(22) AryC,
(23) HetC,
(24) HetT,
(25) C₁₋₆ alkyl substituted with CycC, AryC, HetC, or HetT,
(26) C₁₋₆ alkenyl substituted with CycC, AryC, HetC, or HetT,
(27) C₁₋₆ alkynyl substituted with CycC, AryC, HetC, or HetT,
(28) L-CycC,
(29) L-AryC,
(30) L-HetC, or
(31) L-HetT;
L is:
(1) C(O),
(2) C(O)—C₁₋₆ alkylene, wherein the C₁₋₆ alkylene is optionally substituted with from 1 to 2 substituents each of which is independently OH, C₁₋₆ haloalkyl, O—C₁₋₆ alkyl, O—C₁₋₆ haloalkyl, CN, NO₂, or N(R^{A})R^{B},
(3) C(O)—C₁₋₆ alkylene-O,
(4) C(O)—C₁₋₆ alkylene-O—C₁₋₆ alkylene,
(5) C(O)—C₁₋₆ alkylene-N(R^{A}),
(6) C(O)—C₁₋₆ alkylene-N(R^{A})—C₁₋₆ alkylene,
(7) C(O)N(R^{A}),
(8) C(O)N(R^{A})—C₁₋₆ alkylene,
(9) C(O)N(R^{A})—C₁₋₆ alkylene-C(O)O,
(10) C(O)N(R^{A})—C₁₋₆ alkylene-C(O)N(R^{A}), or
(11) S(O)₂;
CycC independently has the same definition as CycA;
AryC independently has the same definition as AryA;
HetC independently has the same definition as HetA;
HetT independently has the same definition as HetR;
R⁵ is H or independently has the same definition as R¹;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
each CycE is independently C₃₋₈ cycloalkyl which is optionally substituted with from 1 to 4 substituents each of which is independently halogen, C₁₋₆ alkyl, OH, O—C₁₋₆ alkyl, C₁₋₆ haloalkyl, or O—C₁₋₆ haloalkyl;
each AryE is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetE is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, N(R^{A})R^{B}, N(R^{A})C(O)N(R^{A})R^{B}, or N(R^{A})CO₂R^{B};
each HetF is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with a total of from 1 to 4 substituents, each of which is independently halogen, CN, C₁₋₆ alkyl, OH, oxo, O—C₁₋₆ alkyl, C₁₋₆ haloalkyl, or O—C₁₋₆ haloalkyl;
each R^{A} is independently H or C₁₋₆ alkyl; and
each R^{B} is independently H or C₁₋₆ alkyl;
and with the proviso that
(A) when R¹ is chloro, R² is AryB and AryB is unsubstituted phenyl or 4-methylphenyl, R³ is H, and R⁵ is H, then R⁴ is not unsubstituted phenyl, and
(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂.

2. The compound according to claim 1, or a pharmaceutically acceptable salt thereof, wherein R¹ is:
(1) halogen,
(2) CN,
(3) NO₂,
(4) N(R^{A})R^{B},
(5) N(R^{A})S(O)₂R^{B},
(6) N(R^{A})C(O)R^{B},
(7) C₁₋₆ alkyl,
(8) C₁₋₆ haloalkyl,
(9) C₂₋₆ alkenyl,
(10) OH,
(11) O—C₁₋₆ alkyl,
(12) O—C₁₋₆ haloalkyl,
(13) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(14) CycA,
(15) AryA,
(16) HetA, or
(17) C₁₋₆ alkyl substituted with CycA, AryA, or HetA; and
R⁵ is H;
and with the proviso that:
(A) when R¹ is chloro, R² is AryB and AryB is unsubstituted phenyl or 4-methylphenyl, and R³ is H, then R⁴ is not unsubstituted phenyl, and
(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂.

3. The compound according to claim 1, or a pharmaceutically acceptable salt thereof, wherein R² is:
(1) AryB,
(2) HetB,
(3) HetS,
(4) C₁₋₆ alkyl substituted with AryB or HetB,
(5) N(R^{A})-AryB, or
(6) N(R^{A})-HetB;
and with the proviso that:
(A) when R¹ is chloro, R² is AryB and AryB is unsubstituted phenyl or 4-methylphenyl, and R³ is H, then R⁴ is not unsubstituted phenyl, and
(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, R³ is H, and R⁵ is H, then R⁴ is not NH₂, or (ii) when R³ is H and R⁵ is other than H, then R⁴ is not NH₂.

4. The compound according to claim 1, or a pharmaceutically acceptable salt thereof, wherein R³ is H;
and with the proviso that:
(A) when R¹ is chloro, R² is AryB and AryB is unsubstituted phenyl or 4-methylphenyl, and R⁵ is H, then R⁴ is not unsubstituted phenyl, and
(B) (i) when R¹ is other than halogen, CN, NO₂, O—C₁₋₆ alkyl, N(R^{A})R^{B}, N(H)S(O)₂—C₁₋₃ alkyl, or N(H)C(O)—C₁₋₃ alkyl, and R⁵ is H, then R⁴ is not NH₂, or (ii) when R⁵ is other than H, then R⁴ is not NH₂.

5. The compound according to claim 1, or a pharmaceutically acceptable salt thereof, wherein R⁴ is:
(1) C₁₋₆ alkyl,
(2) C₁₋₆ alkyl substituted with O—C₁₋₆ alkyl, O—C₁₋₆ haloalkyl, N(R^{A})R^{B}, C(O)N(R^{A})R^{B}, C(O)R^{A}, CO₂R^{A}, or OC(O)N(R^{A})R^{B},
(3) C₁₋₆ haloalkyl,
(4) C(O)—C₁₋₆ alkyl,
(5) C(O)—C₁₋₆ alkylene-O—C₁₋₆ alkyl,
(6) C(O)—C₁₋₆ alkylene-O(C═O)—C₁₋₆ alkyl,
(7) C(O)—C₁₋₆ alkylene-C(O)O—C₁₋₆ alkyl,
(8) C(O)—C₁₋₆ alkylene-N(R^{A})R^{B},
(9) C(O)—C₁₋₆ alkylene-N(R^{A})—C₂₋₆ alkylene-OH, with the proviso that the OH is not attached to the carbon in C₂₋₆ allylene that is directly attached to the rest of the molecule,
(10) C(O)—C₁₋₆ alkylene-N(R^{A})—C₁₋₆ alkylene-N(R^{A})R^{B},
(11) C(O)N(R^{A})R^{B},
(12) C(O)N(R^{A})—C₁₋₆ alkylene-N(R^{A})R^{B},
(13) C(O)N(R^{A})—C₁₋₆ alkylene-C(O)—O—C₁₋₆ alkyl,
(14) CycC,
(15) AryC,
(16) HetC,
(17) HetT,
(18) C₁₋₆ alkyl substituted with CycC, AryC, HetC, or HetT
(19) L-CycC,
(20) L-AryC,
(21) L-HetC, or
(22) L-HetT; and
L is:
(1) C(O),
(2) C(O)—C₁₋₆ allylene, wherein the C₁₋₆ alkylene is optionally substituted with from 1 to 2 substituents each of which is independently OH, C₁₋₆ haloalkyl, O—C₁₋₆ alkyl, or O—C₁₋₆ haloalkyl,
(3) C(O)—C₁₋₆ alkylene-O,
(4) C(O)—C₁₋₆ alkylene-O—C₁₋₆ alkylene,
(5) C(O)—C₁₋₆ alkylene-N(R^{A}),
(6) C(O)—C₁₋₆ alkylene-N(R^{A})—C₁₋₆ alkylene,
(7) C(O)N(R^{A}), or
(8) C(O)N(R^{A})—C₁₋₆ alkylene;
and with the proviso that:
(A) when R¹ is chloro, R² is AryB and AryB is unsubstituted phenyl or 4-methylphenyl, R³ is H, and R^{S} is H, then R⁴ is not unsubstituted phenyl.

6. The compound according to claim 1, or a pharmaceutically acceptable salt thereof, wherein:
R¹ is:
(1) Cl, Br, or F,
(2) CN,
(3) NO₂,
(4) N(H)—C₁₋₄ alkyl,
(5) N(C₁₋₄ alkyl)₂,
(6) N(H)S(O)₂—C₁₋₄ alkyl,
(7) N(C₁₋₄ allyl)S(O)₂—C₁₋₄ alkyl,
(8) N(H)C(O)—C₁₋₄ alkyl,
(9) N(C₁₋₄ alkyl)C(O)—C₁₋₄ alkyl,
(10) C₁₋₄ alkyl,
(11) C₁₋₄ haloalkyl,
(12) CH═CH₂,
(13) OH,
(14) O—C₁₋₄ alkyl,
(15) O—C₁₋₄ haloalkyl,
(16) C₁₋₄ alkyl substituted with OH, O—C₁₋₄ alkyl, CN, NO₂, N(H)—C₁₋₄ alkyl, or N(C₁₋₄ alkyl)₂,
(17) CycA,
(18) AryA,
(19) HetA, or
(20) C₁₋₄ alkyl substituted with CycA, AryA, or HetA;
R² is
(1) C₁₋₄ alkyl,
(2) C₁₋₄ haloalkyl,
(3) C₁₋₄ alkyl substituted with OH, O—C₁₋₄ alkyl, O—C₁₋₄ fluoroalkyl, CN, NO₂, N(H)—C₁₋₄ alkyl, or N(C₁₋₄ alkyl)₂,
(4) CycB,
(5) AryB,
(6) HetB,
(7) HetS,
(8) C₁₋₄ alkyl substituted with CycB, AryB, HetB, or HetS,
(9) N(H)—C₁₋₄
(10) N(H)—C₁₋₄ alkyl, wherein the C₁₋₄ alkyl is substituted with OH, O—C₁₋₄ alkyl, O—C₁₋₄ fluoroalkyl, CN, NO₂, N(H)—C₁₋₄ alkyl, or N(C₁₋₄ alkyl)₂, with the proviso that the OH, O—C₁₋₄ alkyl, or O—C₁₋₄ fluoroalkyl is not attached to the carbon in C₁₋₄ alkyl that is directly attached to the rest of the molecule,
(11) N(H)-CycB,
(12) N(H)-AryB,
(13) N(H)-HetB, or
(14) N(H)—C₁₋₆ alkyl, wherein the alkyl is substituted with CycB, AryB, HetB, or HetS;
R³ is H;
R⁴ is:
(1) C(O)—C₁₋₄ alkyl,
(2) C(O)—(CH₂)₁₋₄—O—C₁₋₄ alkyl,
(3) C(O)—(CH₂)₁₋₄—O(C═O)—C₁₋₄ alkyl,
(4) C(O)—(CH₂)₁₋₄—C(O)O—C₁₋₄ alkyl,
(5) C(O)—(CH₂)₁₋₄—N(H)—C₁₋₄ alkyl,
(6) C(O)—(CH₂)₁₋₄—N(C₁₋₄ alkyl)₂,
(7) C(O)—(CH₂)₁₋₄—N(H)—(CH₂)₂₋₅OH,
(8) C(O)—(CH₂)₁₋₄—N(H)—(CH₂)₁₋₄—N(H)—C₁₋₄ alkyl,
(9) C(O)—(CH₂)₁₋₄—N(H)—(CH₂)₁₋₄—N(C₁₋₄ alkyl)₂,
(10) C(O)N(H)—C₁₋₆ alkyl,
(11) C(O)N(C₁₋₄ alkyl)₂,
(12) C(O)N(H)—(CH₂)₁₋₄—N(H)—C₁₋₄ alkyl,
(13) C(O)N(H)—(CH₂)₁₋₄—N(C₁₋₄ alkyl)₂,
(14) C(O)N(H)—(CH₂)₁₋₄—C(O)—O—C₁₋₄ alkyl,
(15) CycC,
(16) AryC,
(17) HetC,
(18) HetT,
(19) CH(CH₃)-CycC, CH(CH₃)-AryC, CH(CH₃)-HetC, or CH(CH₃)-HetT
(20) (CH₂)₁₋₄-CycC, (CH₂)₁₋₄-AryC, (CH₂)₁₋₄-HetC, or (CH₂)₁₋₄-HetT
(21) L-CycC,
(22) L-AryC,
(23) L-HetC, or
(24) L-HetT; and
L is:
(1) C(O),
(2) C(O)—(CH₂)₁₋₄, wherein the (CH₂)₁₋₄ is optionally substituted with from 1 to 2 substituents each of which is independently OH, CF₃, O—C₁₋₄ alkyl, or OCF₃,
(3) C(O)—(CH₂)₁₋₄—O,
(4) C(O)—(CH₂)₁₋₄—O—(CH₂)₁₋₄,
(5) C(O)—(CH₂)₁₋₄—O—CH(CH₃),
(6) C(O)—(CH₂)₁₋₄—N(H),
(7) C(O)—(CH₂)₁₋₄—N(C₁₋₄ alkyl),
(8) C(O)—(CH₂)₁₋₄—N(H)—(CH₂)₁₋₄,
(9) C(O)—(CH₂)₁₋₄—N(C₁₋₄ alkyl)-(CH₂)₁₋₄,
(10) C(O)—(CH₂)₁₋₄—N(H)—CH(CH₃),
(11) C(O)—(CH₂)₁₋₄—N(C₁₋₄ alkyl)-CH(CH₃),
(12) C(O)N(H),
(13) C(O)N(C₁₋₄ alkyl),
(14) C(O)N(H)—(CH₂)₁₋₄, or
(15) C(O)N(C₁₋₄ alkyl)-(CH₂)₁₋₄;
R⁵ is H;
CycA is C₃₋₆ cycloalkyl which is optionally substituted with a total of from 1 to 4 substituents, wherein:
(i) from zero to 4 substituents are each independently:
(1) Cl, Br, or F,
(2) CN,
(3) C₁₋₄ alkyl,
(4) OH,
(5) O—C₁₋₄ alkyl, or
(6) C₁₋₄ haloalkyl, and
(ii) from zero to 1 substituent is AryE, HetE, CH₂-AryE, or CH₂-HetE;
AryA is phenyl or naphthyl, wherein the phenyl or naphthyl is optionally substituted with a total of from 1 to 5 substituents, wherein:
(i) from zero to 5 substituents are each independently:
(1) C₁₋₄ allyl,
(2) O—C₁₋₄ alkyl,
(3) C₁₋₄ haloalkyl,
(4) O—C₁₋₄ haloalkyl,
(5) OH,
(6) halogen,
(7) CN,
(8) NO₂,
(9) NH₂,
(10) N(H)—C₁₋₄ alkyl,
(11) N(C₁₋₄ alkyl)₂,
(12) C(O)NH₂,
(13) C(O)N(H)—C₁₋₄ alkyl,
(14) C(O)N(C₁₋₄ alkyl)₂,
(15) C(O)—C₁₋₄ alkyl,
(16) CO₂—C₁₋₄ alkyl,
(17) S—C₁₋₄ alkyl,
(18) S(O)—C₁₋₄ alkyl,
(19) SO₂—C₁₋₄ alkyl,
(20) SO₂NH₂,
(21) SO₂N(H)—C₁₋₄ alkyl,
(22) SO₂N(C₁₋₄ alkyl)₂,
(23) SO₂N(H)C(O)—C₁₋₄ alkyl,
(24) SO₂N(C₁₋₄ allyl)C(O)—C₁₋₄ alkyl,
(25) N(H)C(O)—C₁₋₄ alkyl, or
(26) N(C₁₋₄ alkyl)C(O)—C₁₋₄ alkyl, and
(ii) from zero to 1 substituent is AryE, HetE, CH₂-AryE, or CH₂-HetE;
HetA is (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing a total of from 1 to 4 heteroatoms independently selected from zero to 4 N atoms, zero to 2 O atoms, and zero to 2 S atoms, wherein either one or both of the rings contain one or more of the heteroatoms, at least one ring is aromatic, each N is optionally in the form of an oxide, and each S in a ring which is not aromatic is optionally S(O) or S(O)₂; wherein the heteroaromatic ring or the bicyclic, fused ring system is optionally substituted with a total of from 1 to 4 substituents, wherein:
(i) from zero to 4 substituents are each independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) Cl, Br, or F,
(7) CN,
(8) C(O)N(H)—C₁₋₄ alkyl,
(9) C(O)N(C₁₋₄ alkyl)₂,
(10) S(O)₂—C₁₋₄ alkyl,
(11) S(O)₂NH₂,
(12) S(O)₂N(H)—C₁₋₄ alkyl, or
(13) S(O)₂N(C₁₋₄ alkyl)₂, and
(ii) from zero to 1 substituent is AryE, HetE, CH₂-AryE, or CH₂-HetE;
CycB and CycC each independently have the same definition as CycA;
AryB and AryC each independently have the same definition as AryA;
HetB and HetC each independently have the same definition as HetA;
HetS is a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring contains a nitrogen atom which is directly attached to the rest of the molecule and optionally contains an additional heteroatom selected from N, O, and S, where the S is optionally oxidized to S(O) or S(O)₂; and wherein the heterocyclic or heterobicyclic ring is optionally substituted with a total of from 1 to 4 substituents, wherein:
(i) from zero to 4 substituents are each independently Cl, Br, F, C₁₋₄ alkyl, OH, oxo, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
HetT is a 4- to 7-membered, saturated or mono-unsaturated heterocyclic ring containing from 1 or 2 heteroatoms independently selected from N, O, and S, where each S is optionally oxidized to S(O) or S(O)₂, and wherein the saturated or mono-unsaturated heterocyclic ring is optionally substituted with a total of from 1 to 4 substituents, wherein:
(i) from zero to 4 substituents are each independently Cl, Br, F, C₁₋₄ alkyl, OH, oxo, C(O)NH₂, C(O)N(H)—C₁₋₄ alkyl, C(O)N(C₁₋₄ alkyl)₂, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
AryE is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, Cl, Br, or F, CN, C(O)N(H)—C₁₋₄ alkyl, C(O)N(C₁₋₄ alkyl)₂, S(O)₂—C₁₋₄ alkyl, S(O)₂NH₂, S(O)₂N(H)—C₁₋₄ alkyl, or S(O)₂N(C₁₋₄ alkyl)₂; and
HetE is a 5- or 6-membered heteroaromatic ring containing from 1 to 4 heteroatoms independently selected from N, O and S, wherein each N is optionally in the form of an oxide, wherein the heteroaromatic ring is optionally substituted with from 1 to 3 substituents each of which is independently Cl, Br, F, CN, NO₂, C₁₋₄ alkyl, C₁₋₄ fluoroalkyl, OH, O—C₁₋₄ alkyl, or O—C₁₋₄ fluoroalkyl;
and with the proviso that:
(A) when R¹ is chloro, and R² is AryB and AryB is unsubstituted phenyl or 4-methylphenyl, then R⁴ is not unsubstituted phenyl.

7. The compound according to claim 6, or a pharmaceutically acceptable salt thereof, wherein:
R¹ is chlorine or bromine;
R² is AryB or HetS;
AryB is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, C(O)N(H)—C₁₋₄ alkyl, C(O)N(C₁₋₄ S(O)₂—C₁₋₄ alkyl, S(O)₂NH₂, S(O)₂N(H)—C₁₋₄ alkyl, or S(O)₂N(C₁₋₄ alkyl)₂;
HetS is a saturated heterocyclic or heterobicyclic ring selected from the group consisting of: 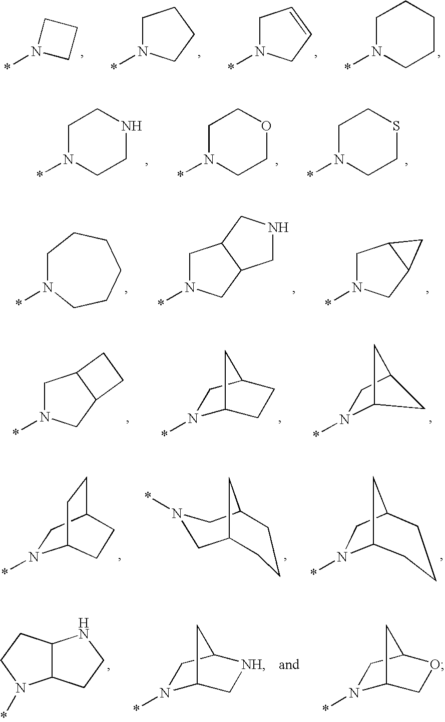
wherein the asterisk denotes the point of attachment of the heterocyclic or heterobicyclic ring to the rest of the molecule, and wherein the heterocyclic or heterobicyclic ring is optionally substituted with a total of from 1 to 4 substituents, each of which is independently C₁₋₄ alkyl, S(O)₂—C₁₋₄ alkyl, O—C₁₋₄ alkyl, C₁₋₄ fluoroalkyl, O—C₁₋₄ fluoroalkyl, oxo, Cl, Br, or F;
R³ is H;
R⁴ is:
(1) C(O)—C₁₋₄ alkyl,
(2) C(O)—(CH₂)₁₋₃—O—C₁₋₄ alkyl,
(3) C(O)—(CH₂)₁₋₃—O(C═O)—C₁₋₄ alkyl,
(4) C(O)—(CH₂)₁₋₃—C(O)O—C₁₋₄ alkyl,
(5) C(O)—(CH₂)₁₋₃—N(H)—C₁₋₄ alkyl,
(6) C(O)—(CH₂)₁₋₃—N(C₁₋₄ alkyl)₂,
(7) C(O)—(CH₂)₁₋₃—N(H)—(CH₂)₂₋₅OH,
(8) C(O)—(CH₂)₁₋₃—N(H)—(CH₂)₁₋₃—N(H)—C₁₋₄ alkyl,
(9) C(O)—(CH₂)₁₋₃—N(H)—(CH₂)₁₋₃—N(C₁₋₄ alkyl)₂,
(10) C(O)NH₂,
(11) C(O)N(H)—C₁₋₄ alkyl,
(12) C(O)N(H)—(CH₂)₂—C₃₋₄ alkyl,
(13) C(O)N(H)—CH₂—C₄ alkyl,
(14) C(O)N(C₁₋₄ alkyl)₂,
(15) C(O)N(H)—(CH₂)₁₋₃—N(H)—C₁₋₄ alkyl,
(16) C(O)N(H)—(CH₂)₁₋₃—N(C₁₋₄ alkyl)₂,
(17) C(O)N(H)—(CH₂)₁₋₃—C(O)—O—C₁₋₄ alkyl,
(18) L-CycC,
(19) L-AryC,
(20) L-HetC, or
(21) L-HetT; and
L is:
(1) C(O),
(2) C(O)—(CH₂)₁₋₃, wherein the (CH₂)₁₋₃ is optionally substituted with from 1 to 2 substituents each of which is independently OH, CF₃, O—C₁₋₄ alkyl, or OCF₃,
(3) C(O)—(CH₂)₁₋₃—O,
(4) C(O)—(CH₂)₁₋₃—O—(CH₂)₁₋₃,
(5) C(O)—(CH₂)₁₋₃—O—CH(CH₃),
(6) C(O)—(CH₂)₁₋₃—N(H),
(7) C(O)—(CH₂)₁₋₃—N(C₁₋₄ allyl),
(8) C(O)—(CH₂)₁₋₃—N(H)—(CH₂)₁₋₃,
(9) C(O)—(CH₂)₁₋₃—N(C₁₋₄ alkyl)-(CH₂)₁₋₃,
(10) C(O)—(CH₂)₁₋₃—N(H)—CH(CH₃),
(11) C(O)—(CH₂)₁₋₃—N(C₁₋₄ alkyl)-CH(CH₃),
(12) C(O)N(H),
(13) C(O)N(C₁₋₄ alkyl),
(14) C(O)N(H)—(CH₂)₁₋₃, or
(15) C(O)N(C₁₋₄ alkyl)-(CH₂)₁₋₃;
CycC is C₃₋₆ cycloalkyl which is optionally substituted with phenyl;
AryC independently has the same definition as AryB;
HetC is (i) a 5- or 6-membered heteroaromatic ring selected from the group consisting of pyrrolyl, thienyl, furanyl, pyrazolyl, imidazolyl, triazolyl, tetrazolyl, oxazolyl, thiazolyl, isoxazolyl, isothiazolyl, oxadiazolyl, pyridinyl, pyrazinyl, and pyrimidinyl or (ii) a bicyclic, fused ring system selected from the group consisting of quinolinyl, isoquinolinyl, quinazolinyl, naphthyridinyl, benzoxazinyl, cinnolinyl, benzofuranyl, 2,3-dihydrobenzo-1,4-dioxinyl, and benzo-1,3-dioxolyl; wherein the heteroaromatic ring or the bicyclic, fused ring system is optionally substituted with a total of from 1 to 3 substituents each of which is independently C₁₋₄ alkyl, O—C₁₋₄ alkyl, C₁ fluoroalkyl, O—C₁₋₄ fluoroalkyl, OH, Cl, Br, or F;
HetT is a saturated or mono-unsaturated heterocyclic ring selected from the group consisting of: 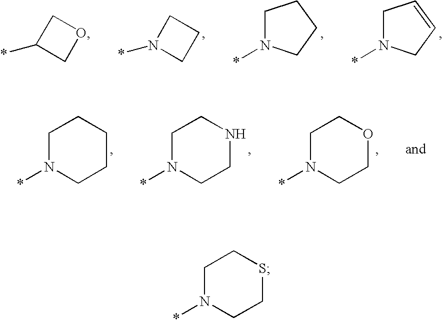
wherein the asterisk denotes the point of attachment of the heterocyclic ring to the rest of the molecule, and wherein the saturated or mono-unsaturated heterocyclic ring is optionally substituted with a total of from 1 to 4 substituents, wherein
(i) from zero to 4 substituents are each independently C₁₋₄ alkyl, C(O)NH₂, C(O)N(H)—C₁₋₄ alkyl, C(O)N(C₁₋₄ alkyl)₂, S(O)₂—C₁₋₄ alkyl, O—C₁₋₄ alkyl, C₁₋₄ fluoroalkyl, O—C₁₋₄ fluoroalkyl, oxo, Cl, Br, or F, and
(ii) from zero to 1 substituent is AryE, HetE, CH₂-AryE, or CH₂-HetE;
AryE is phenyl which is optionally substituted with from 1 to 3 substituents each of which is independently C₁₋₄ alkyl, O—C₁₋₄ alkyl, CF₃, OCF₃, Cl, Br, or F; and
HetE is pyridinyl which is optionally substituted with from 1 to 3 substituents each of which is independently Cl, Br, F, CN, NO₂, C₁₋₄ alkyl, CF₃, OH, O—C₁₋₄ alkyl, or OCF₃.

8. The compound according to claim 7, or a pharmaceutically acceptable salt thereof, wherein:
R¹ is chlorine; and
R² is AryB; and AryB is phenyl which is optionally substituted with from 1 to 3 substituents each of which is independently C₁₋₄ alkyl, O—C₁₋₄ alkyl, CF₃, OCF₃, OH, Cl, Br, F, CN, C(O)N(H)CH₃, C(O)N(CH₃)₂, S(O)₂CH₃, S(O)₂NH₂, S(O)₂N(H)CH₃, or S(O)₂N(CH₃)₂.

9. The compound according to claim 7, or a pharmaceutically acceptable salt thereof, wherein:
R¹ is bromine; and
R² is

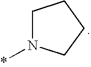

10. A compound, or a pharmaceutically acceptable salt thereof, selected from the group consisting of:
2-(4-chlorophenoxy)-N-[5-chloro-3-(phenylsulfonyl)-1H-indol-2-yl]acetamide;
N-[5-chloro-3-(phenylsulfonyl)-1H-indol-2-yl]-N′-(2-fluorophenyl)urea;
N-[5-chloro-3-(phenylsulfonyl)-1H-indol-2-yl]cyclopropanecarboxamide;
2-{[5-chloro-3-(phenylsulfonyl)-1H-indol-2-yl]amino}-2-oxoethyl acetate;
2-(benzyloxy)-N-[5-chloro-3-(phenylsulfonyl)-1H-indol-2-yl]acetamide;
N-[5-chloro-3-(phenylsulfonyl)-1H-indol-2-yl]propanamide;
N-[5-chloro-3-(phenylsulfonyl)-1H-indol-2-yl]-3-phenoxypropanamide;
N-[5-chloro-3-(phenylsulfonyl)-1H-indol-2-yl]cyclobutanecarboxamide;
N-[5-chloro-3-(phenylsulfonyl)-1H-indol-2-yl]-2,3-dihydro-1-benzofuran-2-carboxamide;
N-[5-chloro-3-(phenylsulfonyl)-1H-indol-2-yl]-2,3-dihydro-1,4-benzodioxine-2-carboxamide;
N¹-[5-chloro-3-(phenylsulfonyl)-1H-indol-2-yl]-N²-cyclopropylglycinamide;
N¹-[5-chloro-3-(phenylsulfonyl)-1H-indol-2-yl]-N²-(pyridin-4-ylmethyl)glycinamide;
N-[5-chloro-3-(phenylsulfonyl)-1H-indol-2-yl]nicotinamide;
N-[5-chloro-3-(phenylsulfonyl)-1H-indol-2-yl]-2-methylpropanamide;
N-[5-chloro-3-(phenylsulfonyl)-1H-indol-2-yl]-2-(4-fluorophenyl)acetamide;
N-[5-chloro-3-(phenylsulfonyl)-1H-indol-2-yl]-2-(3,3-difluoropiperidin-1-yl)acetamide;
N-[5-chloro-3-(phenylsulfonyl)-1H-indol-2-yl]-2,4-difluorobenzamide;
N-[5-chloro-3-(phenylsulfonyl)-1H-indol-2-yl]-2-fluorobenzamide;
N-[5-chloro-3-(phenylsulfonyl)-1H-indol-2-yl]isonicotinamide;
N¹-[5-chloro-3-(phenylsulfonyl)-1H-indol-2-yl]-N¹-ethylglycinamide;
N-[5-chloro-3-(phenylsulfonyl)-1H-indol-2-yl]-4-cyanobenzamide;
N²-benzyl-N¹-[5-chloro-3-(phenylsulfonyl)-1H-indol-2-yl]glycinamide;
N-[5-chloro-3-(phenylsulfonyl)-1H-indol-2-yl]-3-methyl-2-furamide;
N-[5-chloro-3-(phenylsulfonyl)-1H-indol-2-yl]-3-fluorobenzamide;
N-[5-chloro-3-(phenylsulfonyl)-1H-indol-2-yl]-2-methylbutanamide;
ethyl N-({[5-chloro-3-(phenylsulfonyl)-1H-indol-2-yl]amino}carbonyl)glycinate;
N-[5-chloro-3-(phenylsulfonyl)-1H-indol-2-yl]benzamide;
N-[5-chloro-3-(phenylsulfonyl)-1H-indol-2-yl]-N′-(3-fluorophenyl)urea;
N¹-[5-chloro-3-(phenylsulfonyl)-1H-indol-2-yl]-N²-(2-furylmethyl)glycinamide;
N-[5-chloro-3-(phenylsulfonyl)-1H-indol-2-yl]-N′-(4-fluorophenyl)urea;
N¹-[5-chloro-3-(phenylsulfonyl)-1H-indol-2-yl]-N²-(pyridin-3-ylmethyl)glycinamide;
N¹-[5-chloro-3-(phenylsulfonyl)-1H-indol-2-yl]-N²-(isoxazol-3-ylmethyl)glycinamide;
N-[5-chloro-3-(phenylsulfonyl)-1H-indol-2-yl]-2-methoxyacetamide;
N-[5-chloro-3-(phenylsulfonyl)-1H-indol-2-yl]-N′-phenylurea;
N¹-[5-chloro-3-(phenylsulfonyl)-1H-indol-2-yl]-N²-(1-pyridin-4-ylethyl)glycinamide;
N-[5-chloro-3-(phenylsulfonyl)-1H-indol-2-yl]-2-(4-pyridin-4-ylpiperidin-1-yl)acetamide;
N¹-[5-chloro-3-(phenylsulfonyl)-1H-indol-2-yl]-N²-(1,3-thiazol-4-ylmethyl)glycinamide;
(2R)—N-[5-chloro-3-(phenylsulfonyl)-1H-indol-2-yl]-3,3,3-trifluoro-2-methoxy-2-phenylpropanamide;
N-[5-chloro-3-(phenylsulfonyl)-1H-indol-2-yl]-2-[4-(pyridin-2-ylmethyl)piperazin-1-yl]acetamide;
N-[5-chloro-3-(phenylsulfonyl)-1H-indol-2-yl]-N-[2-(trifluoromethyl)phenyl]urea;
N¹-[5-chloro-3-(phenylsulfonyl)-1H-indol-2-yl]-N²-[(1-methyl-1H-imidazol-2-yl)methyl]glycinamide;
N-[5-chloro-3-(phenylsulfonyl)-1H-indol-2-yl]-N′-(3-methylbenzyl)urea;
N-[5-chloro-3-(phenylsulfonyl)-1H-indol-2-yl]-N-cyclopentylurea;
N¹-[5-chloro-3-(phenylsulfonyl)-1H-indol-2-yl]-N²-[(3-methyloxetan-3-yl)methyl]glycinamide;
N-(sec-butyl)-N′-[5-chloro-3-(phenylsulfonyl)-1H-indol-2-yl]urea;
N-[5-chloro-3-(phenylsulfonyl)-1H-indol-2-yl]cyclopentanecarboxamide;
N-butyl-N¹-[5-chloro-3-(phenylsulfonyl)-1H-indol-2-yl]urea;
N-[5-chloro-3-(phenylsulfonyl)-1H-indol-2-yl]-N′-(2-phenylethyl)urea;
N-[5-chloro-3-(phenylsulfonyl)-1H-indol-2-yl]-N-(3-fluorobenzyl)urea;
N-[5-chloro-3-(phenylsulfonyl)-1H-indol-2-yl]-N²-(3-methoxybenzyl)glycinamide;
N-[5-chloro-3-(phenylsulfonyl)-1H-indol-2-yl]-N′-(4-fluorobenzyl)urea;
N-[5-chloro-3-(phenylsulfonyl)-1H-indol-2-yl]-2-[4-(methylsulfonyl)piperazin-1-yl]acetamide;
N¹-[5-chloro-3-(phenylsulfonyl)-1H-indol-2-yl]-N²-(1-pyridin-3-ylethyl)glycinamide;
N¹-[5-chloro-3-(phenylsulfonyl)-1H-indol-2-yl]-N²-(5-hydroxypentyl)glycinamide;
N-[5-chloro-3-(phenylsulfonyl)-1H-indol-2-yl]-2-(3-pyridin-2-ylpyrrolidin-1-yl)acetamide;
N-[5-chloro-3-(phenylsulfonyl)-1H-indol-2-yl]-N′-cyclohexylurea;
N-[5-chloro-3-(phenylsulfonyl)-1H-indol-2-yl]-N′-(2-phenylcyclopropyl)urea;
2-{[5-chloro-3-(phenylsulfonyl)-1H-indol-2-yl]amino}-2-oxo-N-(1-phenylethyl)ethanamine;
N-[5-chloro-3-(phenylsulfonyl)-1H-indol-2-yl]cyclohexanecarboxamide;
N-[5-chloro-3-(phenylsulfonyl)-1H-indol-2-yl]-2-(4-methylpiperazin-1-yl)acetamide;
N-[5-chloro-3-(phenylsulfonyl)-1H-indol-2-yl]-N′-isopropylurea;
N-[5-chloro-3-(phenylsulfonyl)-1H-indol-2-yl]-2-furamide;
N¹-[5-chloro-3-(phenylsulfonyl)-1H-indol-2-yl]-N²-ethyl-N²-(pyridin-4-ylmethyl)glycinamide;
N-[5-chloro-3-(phenylsulfonyl)-1H-indol-2-yl]-2-phenoxyacetamide;
N-[5-chloro-3-(phenylsulfonyl)-1H-indol-2-yl]-N′-(3,5-difluorophenyl)urea;
N-[5-chloro-3-(phenylsulfonyl)-1H-indol-2-yl]-2-[4-(5-methoxypyridin-2-yl)piperazin-1-yl]acetamide;
N-[5-chloro-3-(phenylsulfonyl)-1H-indol-2-yl]-N′-[3-(trifluoromethyl)phenyl]urea; and
N′-(2-{[5-chloro-3-(phenylsulfonyl)-1H-indol-2-yl]amino}-2-oxoethyl)-N,N-diethylethane-1,2-diamine.

11. A compound, or a pharmaceutically acceptable salt thereof, selected from the group consisting of:
N-[5-bromo-3-(pyrrolidin-1-ylsulfonyl)-1H-indol-2-yl]-N′-(3-fluorobenzyl)urea;
N-[5-bromo-3-(pyrrolidin-1-ylsulfonyl)-1H-indol-2-yl]-N′-(3-chlorobenzyl)urea;
N-benzyl-N-[5-bromo-3-(pyrrolidin-1-ylsulfonyl)-1H-indol-2-yl]urea;
N-[5-bromo-3-(pyrrolidin-1-ylsulfonyl)-1H-indol-2-yl]-N′-phenylurea;
N-[5-bromo-3-(pyrrolidin-1-ylsulfonyl)-1H-indol-2-yl]-N′-isopropylurea;
N-[5-bromo-3-(pyrrolidin-1-ylsulfonyl)-1H-indol-2-yl]-N′-pyridin-2-ylurea;
N-[5-bromo-3-(pyrrolidin-1-ylsulfonyl)-1H-indol-2-yl]-N′-cyclopropylurea;
N-[5-bromo-3-(pyrrolidin-1-ylsulfonyl)-1H-indol-2-yl]-N′-(2,6-difluorophenyl)urea;
N-[5-bromo-3-(pyrrolidin-1-ylsulfonyl)-1H-indol-2-yl]-N′-cyclopentylurea;
N-[5-bromo-3-(pyrrolidin-1-ylsulfonyl)-1H-indol-2-yl]-N-(2-hydroxybenzyl)urea;
N-[5-bromo-3-(pyrrolidin-1-ylsulfonyl)-1H-indol-2-yl]-N-(pyridin-2-ylmethyl)urea;
N-[5-bromo-3-(pyrrolidin-1-ylsulfonyl)-1H-indol-2-yl]-N-(pyridin-3-ylmethyl)urea;
N-[5-bromo-3-(pyrrolidin-1-ylsulfonyl)-1H-indol-2-yl]N′-ethylurea;
N-[5-bromo-3-(pyrrolidin-1-ylsulfonyl)-1H-indol-2-yl]-N′-(1,3-thiazol-5-ylmethyl)urea;
N-[5-bromo-3-(pyrrolidin-1-ylsulfonyl)-1H-indol-2-yl]pyrrolidine-1-carboxamide;
N-[5-bromo-3-(pyrrolidin-1-ylsulfonyl)-1H-indol-2-yl]-N′-(2-phenylethyl)urea;
N-[5-bromo-3-(pyrrolidin-1-ylsulfonyl)-1H-indol-2-yl]-N′-(pyridin-4-ylmethyl)urea;
N¹-[5-bromo-3-(pyrrolidin-1-ylsulfonyl)-1H-indol-2-yl]piperidine-1,3-dicarboxamide;
N-[5-bromo-3-(pyrrolidin-1-ylsulfonyl)-1H-indol-2-yl]-N′-(2-pyridin-2-ylethyl)urea;
N-[5-bromo-3-(pyrrolidin-1-ylsulfonyl)-1H-indol-2-yl]urea;
N-[5-bromo-3-(pyrrolidin-1-ylsulfonyl)-1H-indol-2-yl]-N′-(3-phenylpropyl)urea;
N-[5-bromo-3-(pyrrolidin-1-ylsulfonyl)-1H-indol-2-yl]-4-methylpiperazine-1-carboxamide;
N-[5-bromo-3-(pyrrolidin-1-ylsulfonyl)-1H-indol-2-yl]-N′-(3,3-dimethylbutyl)urea;
N-(2-anilinoethyl)-N′[5-bromo-3-(pyrrolidin-1-ylsulfonyl)-1H-indol-2-yl]urea;
N-[5-bromo-3-(pyrrolidin-1-ylsulfonyl)-1H-indol-2-yl]-N′-[3-(dimethylamino)propyl]urea;
N-[5-bromo-3-(pyrrolidin-1-ylsulfonyl)-1H-indol-2-yl]-N′-(2-chloro-6-fluorobenzyl)urea; and
N-[5-bromo-3-(pyrrolidin-1-ylsulfonyl)-1H-indol-2-yl]azetidine-1-carboxamide.

12. A pharmaceutical composition comprising an effective amount of a compound according to any one of claims 1 to 11, or a pharmaceutically acceptable salt thereof, and a pharmaceutically acceptable carrier.

13. A pharmaceutical combination which is (i) a compound according to any one of claims 1 to 11, or a pharmaceutically acceptable salt thereof, and (ii) an HIV infection/AIDS antiviral agent selected from the group consisting of HIV protease inhibitors, nucleoside HIV reverse transcriptase inhibitors, and HIV integrase inhibitors; wherein the compound of (i) or its pharmaceutically acceptable salt and the HIV infection/AIDS antiviral agent of (ii) are each employed in an amount that renders the combination effective for the treatment or prophylaxis of HIV infection or the treatment or prophylaxis or delay in the onset of AIDS.

14. A method for the inhibition of HIV reverse transcriptase, the treatment or prophylaxis of HIV infection, or the treatment or prophylaxis or delay in the onset of AIDS, wherein the method comprises administering to a subject in need thereof an effective amount of a compound of Formula I, or a pharmaceutically acceptable salt thereof, as defined in any one of claims 1 to 11, except that proviso A in the definition of the compound of Formula I is not applied.

15. Use of a compound of Formula I, or a pharmaceutically acceptable salt thereof, as defined in any one of claims 1 to 11, except that proviso A in the definition of the compound of Formula I is not applied, in the inhibition of HIV reverse transcriptase, the treatment or prophylaxis of HIV infection, or the treatment or prophylaxis or delay in the onset of AIDS in a subject in need thereof.

16. A compound of Formula I as defined in any one of claims 1 to 11, or a pharmaceutically acceptable salt thereof, except that proviso A in the definition of the compound of Formula I is not applied, for use in the preparation of a medicament for the inhibition of HIV reverse transcriptase, the treatment or prophylaxis of DIV infection, or the treatment or prophylaxis or delay in the onset of AIDS in a subject in need thereof.
